(19) United States
(12) Patent Application Publication
(10) Pub. No.: US 2010/0330043 A1
(43) Pub. Date: Dec. 30, 2010

(54) GPR125 AS A MARKER FOR STEM AND PROGENITOR CELLS AND METHODS USE THEREOF

(75) Inventors: Shahin Rafii, New York, NY (US); Sergey V. Shmelkov, New York, NY (US); Marco Seandel, New York, NY (US); Sai H. Chavala, Durham, NC (US)

Correspondence Address: WILMERHALE/NEW YORK, 399 PARK AVENUE, NEW YORK, NY, 10022, US

(73) Assignees: CORNELL RESEARCH FOUNDATION, INC., Ithaca, NY (US); SLOAN-KETTERING INSTITUTE FOR CANCER RESEARCH, New York, NY (US)

(21) Appl. No.: 12/446,730

(22) PCT Filed: Oct. 23, 2007
(86) PCT No.: PCT/US07/82184

§ 371 Date: Nov. 24, 2009

Related U.S. Application Data
(60) Provisional application No. 60/853,715, filed on Oct. 23, 2006.
(60) Provisional application No. 60/964,253, filed on Aug. 10, 2007.

(51) Int. Cl. A61K 35/12; C12N 5/074; G01N 33/53; A61P 35/00

(52) U.S. Cl. 424/93.7; 435/325; 435/352; 435/363; 435/366; 435/351; 435/350; 435/368; 435/371; 435/7.21

(57) ABSTRACT

The present invention relates to GPR1 25 as a marker of stem and progenitor cells, including multipotent adult spermatogonial-derived stem cells (MASCs), spermatogonial stem and progenitor cells, skin stem or progenitor cells, intestinal stem or progenitor cells, neural stem or progenitor cells, and cancer stem cells. The invention provides, inter alia, methods for enriching or isolating GPR125-positive stem or progenitor cells, methods for detecting GPR125-positive stem or progenitor cells, methods for culturing GPR125-positive stem or progenitor cells, purified GPR125-positive stem or progenitor cells, therapeutic compositions containing purified GPR125-positive stem or progenitor cells, methods for targeting therapeutic agents to GPR125-positive stem and progenitor cells, and methods of treatment comprising administering GPR125-positive stem and progenitor cells, or differentiated cells derived therefrom, to subjects in need thereof. The present invention also provides methods of detecting cancer cells based on GPR1 25 expression, and methods of targeting therapeutic agents to cancer cells to GPR125-positive cancer cells.

This invention was supported, in part, by NIH grant R01-HL075234 to Dr. Shahin Rafii, and a NIH T32 Institutional Research Training Grant covering Dr. Marco Seandel. Therefore, the U.S. government has certain rights to this invention. For the purposes of the U.S. and other PCT contracting states that permit incorporation by reference only, all publications, patent applications, patents, and other references mentioned herein are incorporated by reference in their entirety.

### FIELD OF THE INVENTION

The present invention relates to markers for stem and progenitor cells, including but not limited to, multipotent adult spermatogonial derived stem cells (referred to as “MASCs”), spermatogonial stem or progenitor cells (referred to as “SSCs”, “SPs” or “SPCs”), skin stem or progenitor cells, intestinal stem or progenitor cells, neural stem or progenitor cells including brain stem or progenitor cells and retinal stem or progenitor cells, and also cancer stem cells, and to methods of use of such stem cell markers, for example in isolating stem or progenitor cells and detecting stem or progenitor cells. The invention also relates, inter alia, to methods of culturing stem or progenitor cells, methods for targeting therapeutic agents to stem and progenitor cells, and methods of treatment comprising administration to subjects in need thereof of stem or progenitor cells, or differentiated cells derived from such stem or progenitor cells.

### BACKGROUND OF THE INVENTION

Stem cell research has the potential to change the face of medical and veterinary science by providing cells that can be used therapeutically to repair specific tissues and organs in the body. The ability to detect, purify, and grow such therapeutically useful stem cells from adult tissues has been hampered by a lack of specific markers. Current evidence indicates that some stem cells may be involved in diseases characterized by excessive cellular proliferation. For example, it has been suggested that “cancer stem cells” may be involved in, or even responsible for, the proliferation of cancer cells in the body. Methods of identifying such over-proliferative stem cells, such as cancer stem cells, and also methods of targeting therapeutic agents to such stem cells, are needed. The present invention addresses these and other needs in the art by providing a marker for stem and progenitor cells, and methods of use thereof.

### SUMMARY OF THE INVENTION

The present invention relates generally to the discovery that the G-protein coupled receptor GPR125 is a marker of stem and progenitor cells, including, but not limited to, multipotent adult spermatogonial derived stem cells (or “MASCs”), spermatogonial stem and progenitor cells, skin stem or progenitor cells, intestinal stem or progenitor cells, neural stem or progenitor cells, and cancer stem cells. The present invention provides, inter alia, methods for enriching or isolating GPR125-positive stem or progenitor cells, methods for detecting GPR125-positive stem or progenitor cells, methods for culturing GPR125-positive stem or progenitor cells, purified GPR125-positive stem or progenitor cells and therapeutic compositions containing such cells. The present invention also provides methods of treatment of subjects, such as human subjects, including, but not limited to, methods of reconstituting or supplementing stem or progenitor cell populations, methods of treating infertility, methods of treating skin conditions, methods of treating intestinal conditions, methods of treating neurological conditions, methods of treating cardiac conditions, methods of treating vascular conditions, methods of treating ischemic conditions, and the like, including autologous stem cell transplantation methods. The present invention provides both methods of treatment that comprise administration of stem or progenitor cells to subjects, and methods of treatment that comprise administration to subjects of differentiated cells derived from stem or progenitor cells. The present invention also provides methods of targeting therapeutic agents to GPR125-positive stem and progenitor cells, such as GPR125-positive cancer cells, and methods of detecting tumors based on the presence of GPR125-positive cancer stem cells.

In one general embodiment, the present invention provides methods for separating, enriching, isolating or purifying stem or progenitor cells from a mixed population of cells, comprising obtaining a mixed population of cells, contacting the mixed population of cells with an agent that binds to GPR125, and separating the subpopulation of cells that are bound by the agent from the subpopulation of cells that are not bound by the agent.

In another general embodiment, the present invention provides a method for detecting stem or progenitor cells in a tissue, tissue sample or cell population based on the presence of GPR125-positive cells. In one such embodiment, the method comprises obtaining a tissue, tissue sample or cell population, contacting the tissue, tissue sample or cell population with an agent that binds to GPR125, and determining whether the agent has bound to the tissue, tissue sample or cell population, wherein binding indicates the presence of stem or progenitor cells and the absence of binding indicates the absence of stem or progenitor cells. In preferred embodiments the agent is an antibody that binds to GPR125. In other embodiments, the present invention provides methods for detecting stem or progenitor cells in a tissue, tissue sample or cell population by determining whether the tissue, tissue sample or cells contain GPR125 mRNA.

In an additional general embodiment, the present invention provides a purified preparation of stem or progenitor cells wherein the cells are positive for GPR125. In one embodiment the invention provides a purified preparation of spermatogonial stem or progenitor cells wherein the cells express GPR125 and at least one gene selected from the group consisting of DAZL, plzf, ret, VASA, integrin alpha 6, Ep-CAM, CD9, GFRa1, glial derived neurotrophic factor (GDNF) and Stra8. In another embodiment, the present invention provides a purified preparation of spermatogonial stem or progenitor cells wherein the cells express GPR125 and at least one gene selected from the group consisting of DAZL, VASA, integrin alpha 6, Ep-CAM, CD9, GFRa1, glial derived neurotrophic factor (GDNF) and Stra8, and do not exhibit detectable expression of at least one gene selected from the group consisting of oct4, nanog, sox2, protamine-1, phosphoglycerate kinase 2, fertilin beta, TP-1 and Sox17. In another embodiment, the present invention provides a purified preparation of MASCs wherein the cells express GPR125 and at least one gene selected from the group consisting of oct4, nanog, and sox2. In another embodiment, the present invention provides a purified preparation of MASCs wherein the cells express GPR125 and at least one gene selected from the group consisting oct4, nanog, and sox2, and do not exhibit detectable expression of at least one gene selected from the group consisting of plzf, ret, stra8, DAZL, gdf3, esg1, and rex1.

In a further general embodiment, the present invention provides therapeutic compositions comprising purified GPR125-positive stem or progenitor cells, or differentiated cells derived therefrom, and a therapeutically acceptable carrier. Such therapeutic compositions are suitable for administration to subjects and for use in accordance with the methods of treatment provided herein.

In an additional general embodiment, the present invention provides methods for culturing GPR125-positive stem and progenitor cells, such as spermatogonial stem or progenitor cells (SPCs) and multipotent adult spermatogonial-derived stem cells (MASCs).

In a further general embodiment, the present invention provides methods for obtaining differentiated cells from GPR125-positive stem and progenitor cells.

In another general embodiment, the present invention provides methods of treatment. Such methods may involve reconstituting or supplementing a cell population in a subject in need thereof, by administering GPR125-positive stem or progenitor cells to the subject, and/or administration of GPR125-positive stem or progenitor cells to subjects in need thereof, and/or administration to subjects of differentiated cells derived GPR125-positive stem or progenitor cells. In a preferred embodiment, the invention provides methods for autologous transplantation, wherein a tissue sample is obtained from a subject, the GPR125-positive stem or progenitor cells from the tissue sample are enriched and expanded in vitro, and then the GPR125-positive stem or progenitor cells, or differentiated cells derived from the GPR125-positive stem or progenitor cells, are administered to the same subject from which the tissue sample was obtained. Such autologous transplantation methods are particularly useful for subjects in need of chemotherapy or radiation therapy, where the tissues samples may be removed from the subject before therapy, and the enriched and expanded GPR125-positive stem or progenitor cells, or cells derived therefrom, may be administered to the subject after therapy.

In an additional general embodiment, the present invention provides a method of targeting a therapeutic agent to a stem or progenitor cell in a subject by conjugating a therapeutic agent to an agent that binds to GPR125 and administering the conjugated agent to the subject. Such methods can be used to target therapeutic agents, such as drugs, to any GPR125-positive cells, such as GPR125-positive cancer stem cells, spermatogonial stem or progenitor cells, skin stem or progenitor cells, intestinal stem or progenitor cells or neural stem or progenitor cells.

In a further general embodiment, the present invention is directed to various methods involving cancer cells. For example, the present invention provides methods for detecting cancer stem cells, methods for detecting tumors, methods for determining whether a subject is likely to develop cancer, and methods for targeting therapeutic agents to cancer cells.

These and other embodiments of the invention are described further in the accompanying Detailed Description, Examples, Drawings, and Claims.

### BRIEF DESCRIPTION OF THE DRAWINGS

To conform to the requirements for PCT applications, many of the figures presented herein are black and white representations of images originally created in color, such as many of those figures based on immunofluorescence microscopy, green fluorescent protein (GFP) labeling, and X-gal (blue) staining. In the below descriptions and the examples, this colored staining is described in terms of its appearance in black and white. For example, X-gal staining which appeared blue in the original appears as a dark stain when presented in black and white. The original color versions of FIGS. 1-14 can be viewed in Seandel et al., Nature (Sep. 20, 2007), Vol. 449, p346-350 (including the accompanying Supplementary Information available in the on-line version of the manuscript available on the Nature web site). For the purposes of the U.S. and other PCT contracting states that permit incorporation by reference, the contents of Seandel et al., Nature (2007), Vol. 449, p346-350, including the accompanying “Supplementary Information,” are herein incorporated by reference.

FIG. 1. Restricted GPR125 expression in adult mouse testis and derivation of multipotent cells from spermatogonial progenitor cells (SPCs). Panels a-c show X-gal staining (dark staining) of adult GPR125βgal mouse testis. Roman numerals in panels c-e denote approximate stages of the seminiferous tubules⁴. Panel d shows quantitation of X-gal staining with tubules grouped as stages IV-V (0.98±0.11 [mean±SE]; n=30 tubules) vs. stages VII-VIII (3.84±0.49; n=28; *p<0.001 by Wilcoxon test). Panel e shows anti-GPR125 staining (arrows) of adult mouse testis. Panel f shows flow cytometry data on freshly dissociated adult GPR125^{lacZ/lacZ} testis. Panel g shows anti-CD34 staining (dark staining) of peritubular/interstitial mouse cells, which remain CD34′ (inset) following in vitro expansion. Panels h-i show highly proliferative GSPC colonies (h) that express plzf after expansion on inactivated CD34⁺mTS. Panel j is a graph showing that GSPC number doubled every ˜2 days. Panels k-l, show appearance of MASCs derived from GPR125⁺ SPCs (GSPCs) following transfer to MEF for expansion and antibody staining, revealing oct-4 expression in the nucleus (right panel in 1). Nuclei are shown by staining of DNA in left panel. Scale bars=50 μm.

FIG. 2. Characterization and multipotent derivatives of GPR125^{lacZ/lacZ} SPC (GSPC) lines. Panel a shows morphology of GPR125βgal GSPC colonies and expression of GPR125 by X-gal staining (dark staining, inset). Panel b shows proliferation of GSPCs in culture. Panel c shows immunolabeling by germ cell markers GCNA (dark staining, left panel), and anti-DAZL (dark staining, right panel). Absence of staining in feeders is denoted by asterisks. Panel d shows expression of GPR125βgal in cloned GSPCs (dark stain), and also tracked by GFP labeling via lentivirus (inset). Panel e shows a bar graph with quantitative PCR data of GPR125^{lacZ/lacZ} GSPCs compared to GPR125^{lacZ/lacZ} total testis. The bars depict fold change compared to total testis in genes associated with GSPCs or differentiating spermatogenic cells. Panels f-h show engraftment of GPR125^{lacZ/lacZ} GSPCs microinjected into busulfan-treated testes. Panel f shows confocal slices (˜1 μm, inset) distinguishing areas with GFP^{bright} spermatogonia along the basement membrane (arrows) from centrally located areas containing smaller, round GFP^{dim} differentiating cells, in the projection of 32 slices. Panel g shows GPR125 expression by X-gal staining (indicated by arrowheads) present in engrafted cells along the basement membrane. Panel h shows differentiation of donor-derived GFP⁺cells and GFP^{neg} non-engrafted tubules (arrowheads denote GFP⁺ spermatids; asterisk denotes non-engrafted tubule). Panel i shows derivation of GPR125⁺ MASCs colonies (dark staining=X-gal, inset) from GSPCs. Panel j shows nuclear labeling by anti-oct4 (dark stain). Panel k shows flow cytometry data for GPR125 expression in GPR125^{lacZ/lacZ} MASCs (right-hand peak) or GSPCs (middle peak) by FDG-staining (mean fluorescence intensity: 22.1 or 18.2, respectively, vs. 2.2 in WT GSPC control (left-hand peak). Scale bars in each panel are 50 μm.

FIG. 3. GPR125βgal MASCs exhibit multipotency and can form functional vessels. Panels a-b show embryoid bodies (EBs) differentiated in vitro and immunolabeled for neuroectoderm (anti-GFAP, panel a); mesoderm (anti-myosin heavy chain (myosin HC, panel b); and endoderm or ectoderm (using anti-HNF3β panel b). Panel c shows X-gal stained GPR125βgal (dark stain). Panels d-f show MASC teratomas formed in NOD-SCID mice. Teratoma histology showing endodermal (panel d), ectodermal (panel e), and mesodermal (panel f) elements. Immunofluorescence staining is shown in the insets for anti-mucin (panel d) and anti-GFAP (panel e). Panels g-h show hole mount embryo X-gal staining (dark stain). Panel g shows an embryonic day 13.5 GPR125βgal MASC chimera formed by blastocyst injection; Panel h shows an embryonic day 14.5 full heterozygous GPR125^{+/lacZ} embryo. Arrowheads denote putative ossification centers. Panel i shows GPR125βgal MASCs differentiated in vitro (22 days) and stained with anti-VE-cadherin—blood vessels can be seen. Panels j-l show cloned MASCs previously transduced in vitro with lentiviral VE-cadherin promoter fragment driving GFP expression form functional teratoma vessels, demonstrated by perfusion with mouse endothelial specific lectin or by the presence of blood in GFP⁺ vessels (black in k-l), inset shows GFP alone). Arrows denote donor-derived vessels. In panels a-c, i, d-e (insets) nuclei are also shown by staining of DNA. The scale bars in each panel are 50 μm.

FIG. 4. GPR125^{lacZ/lacZ} MASCs bear an expression profile different from mouse embryonic stem cells. Panels a-b show data from quantitative PCR experiments comparing expression of relevant genes in vitro in GPR125^{lacZ/lacZ} MASCs vs. wild type ESCs, GPR^{lacZ/lacZ} GSPCs, and MEFs. Panel c is a Venn diagram illustrating transcripts unique or common to GSPCs, MASCs, and ESCs.

FIG. 5. Description of engineered GPR125-LacZ in the native GPR125 locus and fusion protein. Panel a: Construct generated using VelociGene® technology, containing lacZ inserted into exon 16 of mouse GPR125. Boxes and vertical lines denote exons. Panel b: Predicted domain structure of wild type GPR125 and C-terminally truncated GPR125 fused to β-galactosidase. ECD1 denotes the first extracellular domain, TM1-7 denotes transmembrane domains 1 to 7, TM1 denotes the first transmembrane domain, and ICD4 denotes the fourth intracellular domain. The mutant protein retains the N-terminal extracellular domain, the first transmembrane domain, and part of the first intracellular loop of GPR125 fused to β-galactosidase.

FIG. 6. Characterization of testicular stroma in vivo and in vitro. Panels a-b: Cryosections of adult human testis stained with a monoclonal anti-CD34 antibody, using biotinylated secondary antibody followed by streptavidin HRP and DAB (dark stain). Panels c-d: Mouse testicular stromal cells were prepared from adult C57B16 mice and expanded in vitro. Mitomycin-C inactivated mouse testicular stromal (MTS) cells in culture were stained with anti-αsmooth muscle actin (c) or anti-vimentin antibody (d).

FIG. 7. Derivation of MASCs from GPR125⁺ spermatogonial progenitors (GSPCs) using mitotically-inactivated adult testicular stroma (MTS). Panel a: Highly proliferative GSPC colonies supported by MTS after mitomycin-C treatment. Panel b: GSPC cell cycle analysis showing ˜30% of cells in S-phase. Panel c: Six passages following derivation from UBC-GFP mice, cultures contained <1% contaminating GFP⁺ putative somatic cells (i.e., >99% of GFP⁺ cells were part of GSPC colonies). Panels d-f: Expression of germ cell markers by GSPCs: GCNA (d), DAZL (e) by immunohistochemistry (IHC; dark staining staining), and MVH (panel f; bright fluoresecent around cell periphery, bright fluoresecent stain in cell centers is GFP) by immunofluorescence (IF). Panel g, GSPC colonies that gave rise to a transitional morphology after >2 weeks following re-plating were selected and transferred to MEF for expansion as putative multipotent cells, referred to as MASCs. See characteristic Scale bars=50 μm.

FIG. 8. Differentiation of ROSA26-LacZ MASCs in vitro and in vivo. Panels a-d: Ectodermal (a), neuroectodermal (b-c), and mesodermal (d) differentiation in vitro. Hatch lines in c delineate rosettes. Panels e-h: Teratomas formed three weeks after injecting 1×10⁶ MASCs that had been expanded on MEFs into NOD-SCID mice, with evidence of endodermal (f-g), ectodermal (e-f), and mesodermal (f, h) tissue formation.

FIG. 9. Flow cytometry for c-kit expression in GPR125-LacZ SPCs and their cell cycle. Panel a: Absence of c-kit expression in GPR125^{lacZ/lacZ} GSPCs in long-term culture (using IgG control and rat anti-c-kit antibodies). Panel b: Cell cycle analysis by flow cytometry showing GPR125^{lacZ/lacZ} GSPCs in culture exhibit ˜30% of cells in S-phase.

FIG. 10. Expression of canonical SSC markers and markers of differentiating spermatogenic cells in GPR125^{lacZ/lacZ} GSPC culture compared to GPR125^{lacZ/lacZ} total testis. Quantitative PCR using total RNA prepared from passage 5 GPR125^{lacZ/lacZ} GSPCs or fresh adult GPR125^{lacZ/lacZ} testicular tissue. Genes were selected based on specificity for either spermatogonial stem cells, differentiating germ cells, or all germ cells. The left-hand bar in each pair of bars denote GSPCs, and the right-hand bar in each pair of bars denote total testis.

FIG. 11. GPR125^{lacZ/lacZ} GSPCs retain in vivo repopulating activity when cultured on mouse testis stroma. GPR125^{lacZ/lacZ} GSPCs that had been labeled in vitro with lentiviral GFP were microinjected into busulphan-treated C57B16 mouse testes and allowed to engraft for varying lengths of time. Bright staining in panels a-d and g-h is from GFP fluorescence. Panel a: Fluorescence stereomicroscopy of colonies at 90 days. Panels b-h: Confocal microscopy of whole seminiferous tubules after 28 (b-c), 66 (d-f), or 90 (g) days of engraftment. Panel h: Cryosection through 90 day colony (arrows indicate sperm tails; asterisks indicate GFP^{negative} non-donor engrafted tubules).

FIG. 12. GPR125^{lacZ/lacZ} GSPCs maintain GPR125 expression after engraftment into donor testes. GPR125^{lacZ/lacZ} GSPCs that had been labeled in vitro with lentiviral GFP were microinjected into busulphan-treated C57B16 mouse testes and allowed to engraft for 90 days before sacrifice. Whole mounted X-gal staining was performed to detect GPR125 expression. Engrafted colonies were identified by GFP fluorescence which appears as brighter patches in panels a and e. X-gal staining can be seen as dark spots in panels a, b, and e. Panels a-b: An engrafted tubule. Panels c-d: A non-grafted tubule. Arrowheads indicate GFP^{bright} cells that co-express GPR125 (as indicated by both bright GFP fluorescence and dark X-gal staining in the same cells). Panel e: Light and fluorescent microscopy and merged images showing co-expression of GPR125 (dark X-gal staining) and GFP (brighter fluorescent patches). The asterisks denote non-engrafted adjacent tubules.

FIG. 13. GPR125^{lacZ/lacZ} MASCs exhibit multi-lineage differentiation in vivo with concurrent lineage-specific down-regulation of GPR125 expression. GPR125^{lacZ/lacZ} MASCs injected subcutaneously in NOD-SCID mice formed teratomas after three to four weeks. Panels a-i: Histochemistry with X-gal (dark staining) and counterstaining of teratoma section with Nuclear Fast Red demonstrated heterogeneous GPR125 expression, with distinct lineages completely lacking staining (as represented by the arrows in panels c, f, h, and i). Original magnification in panels a-c is 100× and in panels d-i is 400×.

FIG. 14. Expression pattern of GPR125 in embryonic day 14.5 (E14.5) GPR125^{+/lacZ} embryos. Heterozygous E14.5 embryos were obtained from mating of homozygous female GPR125^{lacZ/lacZ} and wild type male mice. Xgal (dark) staining revealed GPR125 expression in most organs. Representative sections are shown as follows: Panel a, epithelial layer (ep) and myenteric plexus (mp) of stomach; Panel b, epithelial layer (ep) and myenteric plexus (mp) of midgut; Panel c, esophagus (es) and aorta (ao); Panel d, metanephros (mn); Panel e, ossification (os) centers of ribs; Panel f, digits; Panel g nasal septum; Panel h, cervical musculature (cm).

FIG. 15 shows GPR125 immunostaining of a testicular germ cell tumor from a first human patient. Positive (dark) staining is seen in abnormal seminiferous tubules (indicated by arrows) adjacent to the tumor, and in the clusters of tumor cells, but not in intervening fibrous stroma (asterisks). Panel a shows the central part of the tumor at 200× magnification. Panel b shows the central part of the tumor at 400× magnification. Panel c shows abnormal tissue adjacent to the tumor at 200× magnification. Panel d shows abnormal tissue adjacent to the tumor at 400× magnification.

FIG. 16 shows GPR125 immunostaining of a testicular germ cell tumor from a second human patient. Positive (dark) staining is seen in abnormal seminiferous tubules (indicated by arrows) adjacent to the tumor, and in the clusters of tumor cells, but not in intervening fibrous stroma (asterisks). Panel a shows the central part of the tumor at 200× magnification. Panel b shows the abnormal tissue adjacent to the tumor at 200× magnification. Panel c shows abnormal tissue adjacent to the tumor at 400× magnification.

FIG. 17 shows GPR125 immunostaining of a testicular germ cell tumor in a third human patient. Positive (dark) staining is seen in clusters of tumor cells, but not in the intervening fibrous stroma (asterisks). Panel a shows the central part of the tumor at 200× magnification. Panel b shows the central part of the tumor at 400× magnification.

FIG. 18 shows an amino acid sequence of human GPR125 (SEQ ID NO: 1).

FIG. 19 shows a nucleotide sequence of the human GPR125 cDNA (SEQ ID NO: 2).

### DETAILED DESCRIPTION OF THE INVENTION

### Introduction

The present invention relates generally to the discovery that the G-protein coupled receptor GPR125 is a marker of stem and progenitor cells, including multipotent adult spermatogonial derived stem cells (referred to as “MASCs”), spermatogonial stem and progenitor cells (referred to interchangeably herein as “SSCs”, “SPs”, or “SPCs”), skin stem or progenitor cells, intestinal stem or progenitor cells, neural stem or progenitor cells, and cancer stem cells. The present invention provides, inter alia, methods for enriching or isolating GPR125-positive stem or progenitor cells, methods for detecting GPR125-positive stem or progenitor cells, methods for culturing GPR125-positive stem or progenitor cells, purified GPR125-positive stem or progenitor cells and therapeutic compositions containing such cells. The present invention also provides methods of treatment of subjects, such as human subjects, including, but not limited to, methods of reconstituting or supplementing stem or progenitor cell populations, methods or treating infertility, methods of treating skin conditions, methods of treating intestinal conditions, methods of treating neurological conditions and autologous stem cell transplantation methods. The present invention also provides methods of obtaining differentiated cells from GPR125-positive stem and progenitor cells, and methods of treatment of subjects, such as human subjects, comprising administering to those subjects differentiated cells or tissues derived from GPR215-positive stem or progenitor cells. The present invention also provides methods of targeting therapeutic agents to GPR125-positive stem and progenitor cells, such as GPR125 positive tumor cells, and methods of detecting tumors based on the presence of GPR125-positive cancer stem cells.

### GPR125

GPR125 is a seven transmembrane spanning G protein-coupled receptor (G-protein-coupled receptor 125), which is also known as PGR21 and tumor endothelial marker 5L (TEM5L). As used herein, the term “GPR125” encompasses any and all homologues, orthologs, derivatives, variants, fragments, polymorphs, or mutant versions of GPR125 that retain the property of being expressed in stem or progenitor cells.

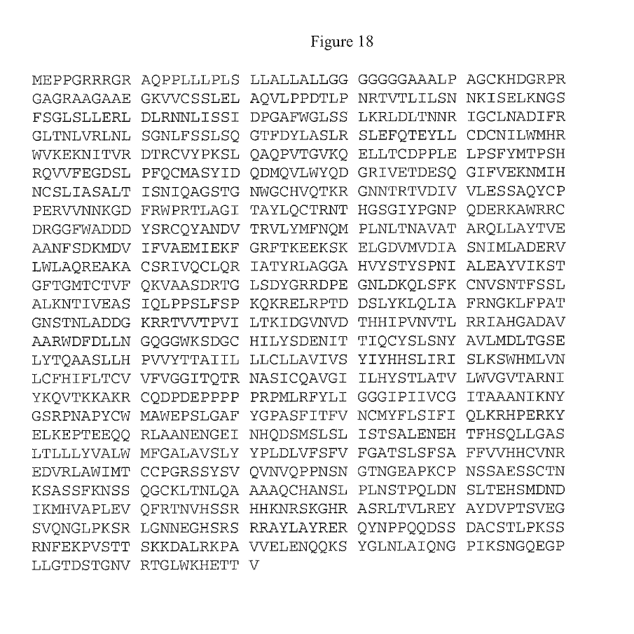

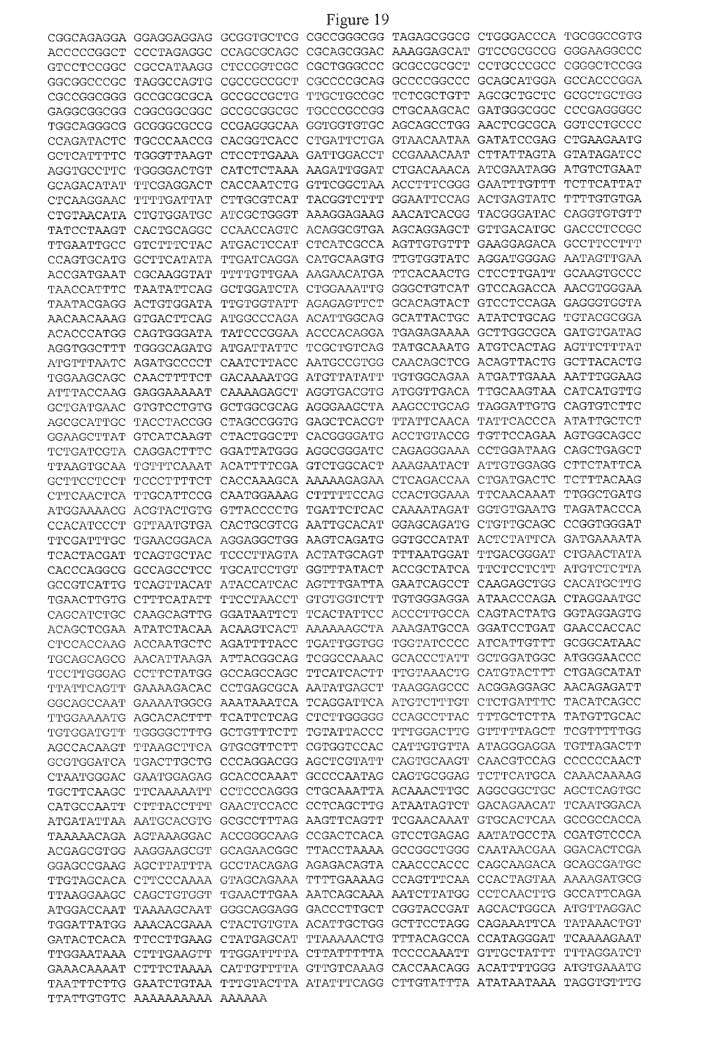

The amino acid sequence of the human GPR125 protein is provided in FIG. 18 (SEQ ID NO: 1; GenBank ID NP: 660333.2). The nucleotide sequence of the human GPR125 mRNA is provided in FIG. 19 (SEQ ID NO: 2; GenBank ID NM: 145290.2). The present invention encompasses, inter alia, a GPR125 protein having the amino acid sequence shown in FIG. 18, or a GPR125 protein that is encoded by the nucleic acid sequence shown in FIG. 19, and homologues, orthologs, derivatives, variants, fragments, polymorphs, or mutant versions thereof. For example, the present invention encompasses, inter alia, the use of any mammalian GPR125 ortholog as a stem cell marker, including, but not limited to, primate, rodent, ovine, bovine, porcine, equine, feline and canine GPR125 orthologs. The present invention also encompasses different polymorphs of GPR125. For example, different individuals from within a given species are likely to contain varying sequences, for example as the result of the presence of single-nucleotide polymorphisms (SNPs).

### GPR125-Positive Stem and Progenitor Cells

The present application relates, in part, to the discovery that GPR125 is a marker of certain stem and progenitor cells. The present invention also relates, in part, to GPR125-positive stem and progenitor cells. GPR125-positive stem and progenitor cells include, but are not limited to spermatogonial progenitor cells (also referred to as “SPs” or “SPCs”) and multipotent adult spermatogonial-derived stem cells (or “MASCs”). Spermatogonial progenitor cells may also be referred to herein, and in the art, as spermatogonial stem cells or “SSCs.” The terms SP, SPC, and SSC, may be used interchangeably herein. MASCs are multipotent cells derived from cultures of SPCs. MASCs have the ability to differentiate into multiple cell types (as described further below and in the Examples). MASCs exhibit other characteristics typical of multipotent cells, such as the ability to contribute to chimeric embryos and the ability to form teratomas in vivo.

### Subjects

As used herein, the term “subject” is used to refer to any animal. In preferred embodiments, the subject is a mammal selected from the group consisting of primates (such as humans and monkeys), rodents, (such as mice, rats and rabbits), ovine species (such as sheep and goats), bovine species (such as cows), porcine species, equine species, feline species and canine species. In a most preferred embodiment, the subject is a human.

### Agents

In certain embodiments, the present invention is directed to agents that bind to GPR125. The agent may be any molecule that has the property of binding to GPR125, without limitation, and, for certain embodiments, such as cell separation and purification embodiments, is preferably an agent that binds to the extracellular domain of GPR125. Thus, the term “agent” includes, but is not limited to, small molecule drugs, peptides, proteins, peptidomimetic molecules and antibodies. The term agent also includes any GPR125 binding molecule that is labeled with a detectable moiety, such as a histological stain, an enzyme substrate, a fluorescent moiety, a magnetic moiety or a radio-labeled moiety. Such “labeled” agents are particularly useful for embodiments involving isolation or purification of GPR125-positive cells, or detection of GPR125-positive cells.

In embodiments where the agent is an antibody, the antibody may be any suitable antibody, such as any polyclonal or monoclonal antibody that binds to GPR125. In certain preferred embodiments, such as cell separation and purification embodiments, the antibody is preferably an antibody that binds to the extracellular domain of GPR125. The term antibody, as used herein also refers to any intact antibody, any antibody fragment that retains the ability to bind to GPR125, and any antibody derivative that retains the ability to bind to GPR125, including, but not limited to, humanized antibody derivatives and fully human antibodies.

In certain embodiments, the agent may be immobilized on a solid support, such as a column, beads, a resin or a microtiter plate. One of skill in the art can readily select a suitable solid support and attach an agent to such a solid support.

### Methods for Enriching, Isolating, or Purifying Stem or Progenitor Cells

The present invention provides methods for separating, enriching, isolating or purifying stem or progenitor cells from a mixed population of cells, comprising obtaining a mixed population of cells, contacting the mixed population of cells with an agent that binds to GPR125, and separating the subpopulation of cells that are bound by the agent from the subpopulation of cells that are not bound by the agent, wherein the subpopulation of cells that are bound by the agent is enriched for GPR125-positive stem or progenitor cells, or contains separated, isolated or purified GPR125-positive stem or progenitor cells.

The methods for separating, enriching, isolating or purifying stem or progenitor cells from a mixed population of cells provided by the present invention may be combined with other methods for separating, enriching, isolating or purifying stem or progenitor cells that are known in the art. For example, the methods described herein may be performed in conjunction with techniques that use other stem cell markers, such as any of the other stem cell markers described herein. For example, an additional selection step may be performed either before, after, or simultaneously with the GPR125 selection step, in which a second agent, such as an antibody, that binds to a second stem cell marker is used. The second stem cell marker may be any stem cell marker known in the art, and/or any of the stem or progenitor cell markers described herein. For example, in one embodiment, the second stem cell marker is selected from the group consisting of alpha-6 integrin, DAZL, plzf, ret, VASA, Ep-CAM, CD9, GFRa1, glial derived neurotrophic factor (GDNF) and Stra8.

The mixed population of cells can be any source of cells from which it is desired to obtain GPR125-positive stem or progenitor cells, including but not limited to a tissue biopsy from a subject, a dissociated cell suspension derived from a tissue biopsy, or a population of cells that have been grown in culture. For example, in one embodiment, the mixed cell population may contain cultured GPR125-positive stein or progenitor cells mixed with other cells, such as spermatogonial stem cells mixed with testicular feeder cells. In preferred embodiments, the mixed population of cells is obtained from a testicular biopsy sample.

The agent used can be any agent that binds to GPR125, as described above. In preferred embodiments, the agent is an antibody that binds to GPR125. In more preferred embodiment, the agent is an antibody that binds to the extracellular domain of GPR125.

There are many cell separation techniques known in the art, and any such technique may be used. For example magnetic cell separation techniques may be used if the agent is labeled with an iron-containing moiety. Cells may also be passed over a solid support that has been conjugated to an agent that binds to GPR125, such that the GPR125-positive cells will be selectively retained on the solid support. Cells may also be separated by density gradient methods, particularly is the agent selected significantly increases the density of the GPR125-positive cells to which it binds. In a preferred embodiment, the agent is a fluorescently labeled antibody against GPR125, and the GPR125-positive stem or progenitor cells are separated from the other cells using fluorescence activated cell sorting (FACs). One of skill in the art can readily perform such cell sorting methods without undue experimentation.

### Methods for Detecting Stem or Progenitor Cells

In a second general embodiment, the present invention provides a method for detecting stem or progenitor cells in a tissue, tissue sample or cell population, wherein the method comprises obtaining a tissue, tissue sample or cell population, contacting the tissue, tissue sample or cell population with an agent that binds to GPR125, and determining whether the agent has bound to the tissue, tissue sample or cell population, wherein binding indicates the presence of stem or progenitor cells and the absence of binding indicates the absence of stem or progenitor cells. In certain embodiments, the amount of agent bound to the tissue, tissue sample or cell population is quantified, wherein the greater the amount of agent that is bound, the greater the number of stem or progenitor cells the tissue, tissue sample or cell population contains. The binding of the agent may also be localized such that specific tissue regions and specific cells types that are positive for GPR125 can be identified.

The agent used can be any agent that binds to GPR125, as described above. In preferred embodiments, the agent is an antibody that binds to GPR125. In more preferred embodiment, the agent is an antibody that binds to the extracellular domain of GPR125. More preferably still, the antibody is labeled with a detectable moiety, such as a histological stain, an enzyme substrate, a fluorescent moiety, a magnetic moiety or a radiolabeled moiety.

There are many cell and protein detection techniques known in the art, and any such techniques may be used. For example, the presence of GPR125-positive cells may be detected by performing immunostaining of tissues, tissue samples, or cells, and detecting the presence of bound antibody. For example, this can be performed using a fluorescently labeled antibody to perform the immunostaining and then using fluorescence microscopy, such as confocal fluorescence microscopy, to detect the labeled cells. Cells labeled with fluorescent antibodies can also be detected by other techniques, including, but not limited to, flow cytometry techniques. Importantly, the agent used may comprise two or more “layers” of agents. For example the agent may consist of a primary antibody that binds to GPR125 but that is not itself labeled with a detectable moiety, and a secondary antibody that binds the primary antibody wherein the secondary antibody is labeled with a detectable moiety. Such multi-layered detection techniques and agents are advantageous in that they may enhance the ability to detect low levels of GPR125 protein by amplifying the amount of detectable moiety that can bind (indirectly) to the GPR125 protein. Any suitable method and any suitable detectable moiety can be used for such immunostaining-based detection methods. Other types of immuno-based detection methods that may be employed include, but are not limited to, Western blotting and immunoprecipiation.

In certain embodiments, the present invention provides methods for detecting stem or progenitor cells in a tissue, tissue sample or cell population by determining whether the tissue, tissue sample or cell contains GPR125 mRNA. The presence of GPR125 mRNA indicates the presence of stem or progenitor cells. Furthermore, the greater the amount of GPR125 mRNA detected, the greater the number of GPR125-positive stem cells there are likely to be in the tissue, tissue sample or cells. There are many suitable techniques known in the art for detection of specific mRNAs and any such method can be used in accordance with the present invention. For example, GPR125 mRNA may be detected by RT PCR, in situ hybridization, Northern blotting and RNAase protection, amongst other methods.

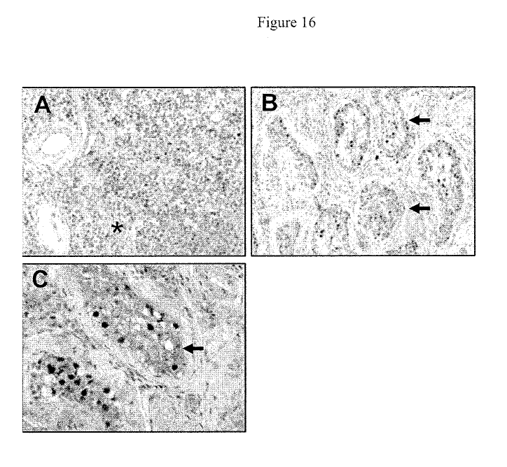

Such methods involve the use of primers and/or probes specific for GPR125. These primers and/or probes may be any nucleotide sequence that binds to a GPR125 mRNA or cDNA. The primers or probes should be of sufficient length to anneal to or hybridize with (i.e. form a duplex with) the GPR125 mRNA or cDNA. Such primers and/or probes may comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 or 100 consecutive nucleotides. In embodiments involving the detection of GPR125 in a human tissue sample, it is preferred that the primers or probes comprise a string of consecutive nucleotides that are complementary to the human GPR125 mRNA or cDNA of FIG. 16 (SEQ ID NO: 2), or that anneal to or hybridize to a human GPR125 mRNA or cDNA under stringent conditions

The primers or probes may be labeled with any suitable molecule and/or label known in the art, including, but not limited to fluorescent tags suitable for use in Real Time PCR amplification, for example TaqMan™, cybergreen, TAMRA and/or FAM probes. The primers or probes may also comprise other detectable non-isotopic labels, such as chemiluminescent molecules, enzymes, cofactors, enzyme substrates or haptens. The primers and/or probes may also be labeled with a radioisotope, such as by incorporation into the primer or probe of a radiolabeled nucleotide, such as a ³²P dNTP.

In preferred embodiments, the hybridization or annealing conditions used are stringent conditions, such that GPR125 mRNAs or cDNAs are detected specifically with minimal background from other mRNAs or cDNAs. As used herein, the phrase “stringent conditions” refers to conditions under which a probe, primer or oligonucleotide will hybridize to GPR125 mRNAs or cDNAs, and can also hybridize to, variant sequences, including allelic or splice variant sequences, orthologs, paralogs, and the like. The precise conditions for stringent hybridization/annealing conditions are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

One of skill in the art can readily select suitable primers or probes for the detection of GPR125 mRNA or cDNA, and can readily use these primers or probes in conjunction with any of the known techniques for mRNA or cDNA detection known in the art. For example, suitable methods are disclosed in Sambrook et al. (2001) Molecular Cloning: A Laboratory Manual, 3rd Ed., Cold Spring Harbor Laboratory, Cold Spring Harbor, N.Y. (“Sambrook”) and Haymes et al., “Nucleic Acid Hybridization: A Practical Approach”, IRL Press, Washington, D.C. (1985), both of which references are incorporated herein by reference.

### Methods of Culturing GPR125-Positive Stem or Progenitor Cells, and Methods of Obtaining Differentiated Cells Therefrom

The present invention provides methods for culturing (and/or enriching or expanding) GPR125-positive stem and progenitor cells. For example, the present invention provides methods of culturing GPR125-positive “SPs” (also referred to as “SPCs” or “SSCs”) and methods of culturing GPR125-positive “MASCs”. The present invention also provides methods of obtaining differentiated cells from GPR125-positive MASCs. Such methods are described below, and are also described in the Examples section of this application. One of skill in the art will recognize that certain modifications or variations of the culture methods described herein can be performed without departing from the spirit of the invention. All such modifications and variations are within the scope of the invention.

### Methods of Culturing, Enriching, or Expanding GPR125-Positive SPs

Suitable methods for culturing SPs are described in the Examples section of this application. Each method involves, as a preliminary step, generating or obtaining a culture of seminiferous tubular cells. These seminiferous tubular cells may be derived from any animal species as desired, such as, for example, humans or mice.

In one embodiment, the method used to culture (and/or enrich or expand) SPs comprises culturing seminiferous tubular cells on a suitable feeder cell layer. Various different types of feeder cell layers are known in the art to be useful for culturing stem and progenitor cells, such as embryonic fibroblast feeder cultures and the like. One of skill in the art can select a suitable feeder layer for use with the methods of the present invention.

In a preferred embodiment, the feeder layer used is a testicular cell feeder layer. In an even more preferred embodiment, the testicular cell feeder layer comprises testicular cells that have been treated with an agent that blocks the cell cycle, or an agent that cross-links DNA or an agent that inhibits RNA synthesis, such as, for example, mitomycin C.

In one embodiment, the method used to culture (and/or enrich or expand) SPs comprises obtaining a sample of seminiferous tubular cells, dissociating the seminiferous tubular cells, plating the dissociated seminiferous tubular cells on matrigel-coated plates, culturing the dissociated seminiferous tubular cells in medium comprising bFGF, EGF, and GDNF, and performing at least 3, or more preferably at least 4, or at least 5, or at least 6, serial passages of the cultured dissociated seminiferous tubular cells onto a mitomycin C-treated testicular cell feeder layer.

In another embodiment, the method used to culture (and/or enrich or expand) SPs comprises obtaining a sample of seminiferous tubular cells, dissociating the seminiferous tubular cells, plating the dissociated seminiferous tubule cells onto a testicular cell feeder layer, culturing the dissociated seminiferous tubule cells on the feeder layer in medium containing StemPro® bFGF, EGF, LIF and GDNF and performing at least 3, or more preferably at least 4, or at least 5, or at least 6, non-enzymatic serial passages of the cultured seminiferous tubule cells onto testicular cell feeder layers.

In yet another embodiment, the method used to culture (and/or enrich or expand) SPs comprises comprising preparing a culture of testicular feeder cells by obtaining a sample of seminiferous tubular cells, dissociating the seminiferous tubular cells, plating the dissociated seminiferous tubular cells onto plates coated with either matrigel or gelatin, and culturing the dissociated seminiferous tubular cells in a suitable growth medium, and then preparing a culture of SPs by obtaining a sample of seminiferous tubular cells, dissociating the seminiferous tubular cells, plating the dissociated seminiferous tubular cells on a layer of the testicular feeder cells, culturing the dissociated seminiferous tubular cells on the feeder cell layers in medium containing StemPro® bFGF, EGF, LIF and GDNF, and performing at least 3, or more preferably at least 4, or at least 5, or at least 6, non-enzymatic serial passages of the cultured cells onto testicular feeder cell layers.

Variations in, and combinations of, each of the above methods can be performed, as will be apparent to those of skill in the art. One of skill in the art can readily perform such culture methods using the above description, and the description provided in the Examples section below, in conjunction with standard cell culture techniques and methods known in the art. See for example, Culture of Animal Cells: A Manual of Basic Technique, 4th Edition (2000) by R. Ian Freshney (“Freshney”), the contents of which are hereby incorporated by reference.

SPCs can be detected and distinguished from the background of testis-derived non-stem cells on the basis of their morphology (see Examples), their characteristic expression profile, and their ability to colonize the testis and reconstitute spermatogenesis in infertile animals, such as in bisulfan-treated mice. SPCs express high levels of of plzf, ret, stra8, and DAZL, in addition to GPR125, but do not express (or express minimal levels of) oct4, nanog, and sox2. Further details of the characteristics of SPCs are provided in the Examples.

### Methods of Culturing, Enriching, or Expanding MASCs

MASCs are multipotent cells derived from SPCs. MASCs have the ability to differentiate into multiple cell types (as described further below and in the Examples). MASCs exhibit other characteristics typical of multipotent cells, such as the ability to contribute to chimeric embryos and the ability to form teratomas in vivo.

MASCs emerge spontaneously from cultures of SPCs. MASCs can be recognized, and distinguished from SPCs, under phase contrast microscopy by their atypical transitional morphology. MASCs have a very high nuclear to cytoplasmic ratio, a large nucleolus, and very little cytoplasm. MASC colonies are highly refractile. Moreover, MASCs morphologically resemble embryonic stem cells, and MASC colonies morphologically resemble embryonic stem cell colonies. Further details of the appearance of MASCs areovided in the Examples, and images of MASC colonies are provided in the Figures. One of skill in the art would readily be able to recognize the emergence of MASCs and MASC colonies.

MASCs may also be recognized by virtue of their expression profile, which differs from that of SPCs. Thus in contrast to SPCs, MASCs express high levels of the markers oct4, nanog, and sox2, and minimal expression of plzf, ret, stra8, and DAZL. Both SPCs and MASCs express GPR125. Unlike ES cells, MASCs exhibit minimal expression of gdf3, esg1, and rex1.

MASCs may also be recognized and distinguished from SPCs by their ability to form embryoid bodies (“EBs”) in vitro. Methods for inducing and detecting EB formation are described in the Examples. Other methods of inducing EB formation are well known in the art, and any such method can be used to confirm the presence of MASCs.

MASCs may also be recognized and distinguished from SPCs by their ability to form teratomas in vivo. Methods for inducing and detecting teratoma formation are described in the Examples. Other methods of inducing and detecting teratoma formation are well known in the art, and any such method can be used to confirm he presence of MASCs.

MASCs may also be recognized and distinguished from SPCs by their ability to contribute to the formation of chimeric embryos in vivo. Methods for producing chimeric embryos are described in the Examples. Other methods of forming chimeric embryos are known in the art, and any such method can be used to confirm in the presence of MASCs.

MASCs may be left in their original culture vessel, i.e. they may continue to be cultured together with SPCs. However, under such conditions, MASCs may spontaneously differentiate into other cell types. In order to obtain cultures of MASCs that may be expanded and that may be maintained in their non-differentiated multipotent state until it is desired to differentiate them, one of more colonies of MASC cells, or a portion of a MASC colony, should be removed from co-culture with SPCs and re-plated in another culture vessel. MASC colonies may be removed using any suitable method known in the art. In a preferred embodiment, one or more MASC colonies is mechanically separated from the culture vessel containing SPCs, such as by using a sterile pasteur pipette or a similar device.

After one or more MASC colonies has been removed, the MASCs should be replated in a suitable culture vessel. MASCs may be cultured in the absence of a feeder layer. For example, feeder-free culture methods that are suitable for culture of other multipotent cells may be used. In preferred embodiments, MASCs are re-plated on a suitable feeder layer. Any suitable feeder layer may be used. For example, several different types of feeder cells are known to be useful for maintaining multipotent stem cells in a non-differentiated state and any such feeder layer can be used. For example, types of feeder layers used to maintain embryonic stem cells in a non-differentiated state may be used. In a preferred embodiment, the MASCs are replated on a feeder layer of embryonic fibroblasts. In a further preferred embodiment, the MASCs are plated on a feeder layer comprising mitomycin-C-inactivated embryonic fibroblasts, such as CF1 mitomycin-C-inactivated mouse embryonic fibroblasts (“MEF”s), which are available commercially from Chemicon or can be obtained from other sources.

The transferred MASCs may be cultured in any suitable medium. For example, culture media known to be useful for maintaining other multipotent cells, such as embryonic stem cells, preferably in an undifferentiated, may be used. In one preferred embodiment, the MASCs are cultured in a medium suitable for culture of SPCs. It has been found that when this culture medium is used but the MASCs are not grown on testicular feeders, the MASCs will remain in an undifferentiated state. Suitable examples of such SPC culture media are provided in the Examples section of this application. In another preferred embodiment, the MASCs are cultured in a medium suitable for growth or embryonic stem cells (“ESCs”). Suitable examples of such ESC culture media are provided in the Examples section of this application. One of skill in the art will recognize that variations in the culture conditions and media can be made. Any such variations may be used so long as the MASCs retain the characteristics desired, such as, for example, proliferative potential, and/or an undifferentiated state, and/or GPR125 expression.

MASCs may proliferate in culture and can be passaged as desired using any suitable method known in the art, at any suitable frequency, and at any suitable dilution. One of skill in the art will readily be able to deter mine suitable passaging conditions. In one preferred embodiment, MASCs are passaged by trypsinization. In another preferred embodiment, MASCs are passaged every 2-4 days. It is preferred that MASCs are passaged onto fresh feeder layers.

Methods of Obtaining Differentiated Cells and Tissues from MASCs, and Identification of Differentiated Cell Types.

As described above and in the Examples, MASCs will spontaneously differentiate into multiple other cell and tissue types under appropriate conditions. For example, if MASCs are co-cultured with SPCs and/or on a feeder layer of testicular stromal cells, they will spontaneously differentiate into multiple other cell types. If MASCs are removed from a feeder layer that is used to keep them in an undifferentiated state, such as a MEF feeder layer, they will spontaneously differentiate into multiple other cell types. If MASCs are placed in a high serum medium, they will spontaneously differentiate into multiple other cell types. Additionally, any of the culture conditions and/or methods used to induce embryonic stem cells to differentiate can be used to induce differentiation of MASCs.

MASCs can spontaneously differentiate into many different types of cells. Teratoma data (see Examples) shows that MASCs are able to differentiate into cells of all three germ cell layers, i.e. endodermal, ectodermal, and mesodermal cell types. Data from various in vivo and in vitro studies (see Examples) shows that MASCs can differentiate into mucin-positive endoderm, GFAP+ neuroectoderm, chondrocytic cells, osteogenic cells, chondrogenic cells, GCNA+ primitive gonad-like cells, myoid cells, vascular endothelial cells (capable of forming functional blood vessels), rhythmically contracting cardiac cells, neurons, gut cells, and skin cells. It is likely that MASCs are also able to differentiate into multiple other types of cells.

The type or types of cells that the MASCs have differentiated into can be determined by a variety of methods, such as by morphological assessment and by the detection of expression of markers associated with those cell types. Expression of such markers may be detected at the mRNA and/or protein levels using standard methods known in the art. Down-regulation of expression of GPR125 and other multipotency markers may also be used as an indicator that differentiation has occurred. Details of how different MASC-derived differentiated cell types may be identified are provided in the Examples section.

### Methods of Preserving GPR125-Positive Stem or Progenitor Cells

In each of the above cell culture embodiments, it is possible to cryogenically freeze and store cells at any step in the process, such as after biopsy, after dissociation of biopsy material, after culture of cells for various periods of time, after obtaining cultures of SPCs, after emergence of MASCs, and after differentiation of MASCs into differentiated cells types, such that the cells may be used at a later time. This is particularly advantageous for the autologous transplantation methods provided herein. Methods of cryogenically freezing and storing cells and tissue samples are well known in the art, and any such method can be used. See, for example, Freshney. Methods of cryogenically freezing the cells of the invention are also provided in the Examples.

### Purified GPR125-Positive Stem or Progenitor Cells

In certain embodiments, the present invention provides purified preparations of GPR125-positive stem or progenitor cells, such as those obtained using the cell separation and/or cell culture methods described above. As used herein the term “purified” does not mean that there can not be any non-GPR125-positive cells present in the preparation. Instead the term “purified” means substantially free of non-GPR125-positive stem or progenitor cells, or pure enough to be safe for administration to a living subject, or pure enough to satisfy the requirements for safety of biologic products laid down by the FDA.

In a preferred embodiment, the invention provides a purified preparation of spermatogonial stem or progenitor cells, that are positive for GPR125.

In the case of SPCs, it is preferred that, in addition to GPR125, the cells are also positive for, or express high levels or, at least one marker selected from the group consisting of DAZL, plzf ret, VASA, integrin alpha 6, Ep-CAM, CD9, GFRa1, glial derived neurotrophic factor (GDNF) and Stra8. More preferably still, the SPCs are positive for, or express high levels of, GPR125 and at least one marker selected from the group consisting of DAZL, plzf ret, VASA, integrin alpha 6, Ep-CAM, CD9, GFRa1, glial derived neurotrophic factor (GDNF) and Stra8, and are negative for, or express minimal levels of, at least one marker selected from the group consisting of protamine-1, phosphoglycerate kinase 2, fertilin beta, TP-1 and Sox17.

In the case of MASCs, it is preferred that, in addition to GPR125, the cells are also positive for, or express high levels of, at least one marker selected from the group consisting of oct4, nanog, and sox2. More preferably still, the MASCs are positive for, or express high levels of, GPR125 and at least one marker selected from the group consisting of oct4, nanog, and sox2, and are negative for, or express minimal levels of, at least one marker selected from the group consisting of plzf, ret, stra8, DAZL, gdf3, esg1, and rex1.

### Therapeutic Compositions Comprising Purified GPR125-Positive Stem or Progenitor Cells

Several embodiments of the invention involve therapeutic compositions comprising purified GPR125-positive stem or progenitor cells (such as GPR125-positive SPCs or GPR125-positive MASCs), or therapeutic compositions comprising differentiated cells derived from GPR125-positive stem or progenitor cells. In preferred embodiments, these compositions comprise a purified preparation GPR125-positive stem or progenitor cells, or a purified preparation of differentiated cells derived from such GPR125-positive stem or progenitor cells, as described above, and a carrier suitable for administration to living subjects, such as humans. In a preferred embodiment the carrier is a physiological saline solution. Other therapeutically acceptable agents may be included if desired. One of skill in the art can readily select suitable agents to be included in the therapeutic compositions depending on the desired outcome.

### Methods of Treatment Using GPR125-Positive Stem or Progenitor Cells

The present invention also provides various methods of treatment. For example, the present invention provides a method of reconstituting or supplementing a cell population in a subject in need thereof, comprising administering to the subject GPR125-positive stem or progenitor cells. In a preferred embodiment, this method comprises obtaining a tissue sample, enriching and expanding the GPR125-positive stem or progenitor cells from the tissue sample in vitro, and then administering the GPR125-positive stem or progenitor cells to the subject. One of skill in the art can readily perform such methods by preparing a therapeutic composition containing GPR125-positive stem cells, as described above, and administering the therapeutic composition to a suitable subject, such as a human patient, using the administration methods of described below.

In preferred embodiments, the present invention provides methods for autologous transplantation, wherein a tissue sample is obtained from a subject, the GPR125-positive stem or progenitor cells from the tissue sample are enriched and expanded in vitro, for example using the methods described above, and then the GPR125-positive stem or progenitor cells are administered to the same subject from which the tissue sample was obtained, for example using the administration methods described below. Such autologous transplantation methods are particularly useful for subjects in need of chemotherapy or radiation therapy, where a tissue sample may be removed from the subject before therapy, and the enriched and expanded GPR125-positive stem or progenitor cells may be administered to the subject after therapy.

In preferred embodiments of the present invention, the GPR125-positive stem or progenitor cells may be multipotent stem cells, spermatogonial stem or progenitor cells, skin stem or progenitor cells, intestinal stem or progenitor cells or neural stem or progenitor cells. Methods of treatment using GPR125-positive skin stem or progenitor cells may be particularly useful when the subject is suffering from, or is at risk of developing, a disease, disorder, or condition affecting the skin or hair follicles, such as skin cancer, burns, traumatic injury to the skin, surgical wounds, aging of the skin, or hair loss. Methods of treatment using GPR125-positive intestinal stem or progenitor cells may be particularly useful when the subject is suffering from, or is at risk of developing, a disease, disorder, or condition affecting the intestinal tract, such as traumatic injury to the intestinal tract or tumors affecting the intestinal tract. Methods of treatment using GPR125-positive neural stem or progenitor cells may be particularly useful when the subject is suffering from, or is at risk of developing, a disease, disorder, or condition affecting the nervous system (including the retina) such as spinal cord injury, traumatic brain injury, a neural tumor, a neurodegenerative disease, Parkinson's disease, Alzheimer's disease, Lewy body dementia, Creutzfeldt-Jakob disease, Huntington disease, multiple sclerosis, traumatic retinal injury, retinopathy, retinoblastoma, a retinal degenerative disease or macular degeneration. Methods of treatment using GPR125-positive multipotent stem cells may be useful for treating a variety of conditions in a variety of tissues. In one embodiment, the GPR125-positive multipotent stem cells differentiate spontaneously into cell types characteristic of the site/tissue where they are administered, such as in response to local cues (such as local cellular interactions and other local factors). For example, it is possible that when GPR125-positive multipotent stem cells are administered to the nervous system they may differentiate into neurons in response to local environmental cues. In another embodiment, the GPR125-positive multipotent stem cells are treated with, or co-administered with an agent that encourages their differentiation into a particular cell type.

One of skill in the art can readily perform such treatment methods by preparing a therapeutic composition containing GPR125-positive stem cells, as described above, and administering the therapeutic composition to a suitable subject, such as a human patient, using the administration methods described below.

### Methods of Treatment Using GPR125-Positive Spermatogonial Stem or Progenitor Cells

In certain embodiments, the present invention provides methods for reconstituting or supplementing spermatogenesis in a subject in need thereof, wherein the method comprises administering to the subject GPR125-positive spermatogonial stem or progenitor cells. In a preferred embodiment, the invention provides a method for reconstituting or supplementing spermatogenesis in a subject in need thereof, comprising obtaining a sample of seminiferous tubular cells, dissociating the seminiferous tubular cells, plating the dissociated seminiferous tubular cells on a layer of testicular feeder cells, culturing the dissociated seminiferous tubular cells on the feeder cells in a medium containing StemPro® bFGF, EGF, LIF and GDNF, performing at least 3, or more preferably at least 4, or at least 5, or at least 6, non-enzymatic serial passages of the cultured cells onto testicular feeder cell layers, separating the GPR125-positive spermatogonial cells from the feeder cells and any other cells present in the culture, and administering the GPR125-positive spermatogonia stem or progenitor cells to the subject.

Such methods are particularly useful for subjects that are infertile or have reduced fertility. The methods may also be useful for subjects who are suffering from, or who are at risk of developing, a disease, disorder, or condition such as a genetic disorder of the Y chromosome, Y chromosome microdeletions, Klinefelters syndrome, testicular cancer, seminoma, idiopathic testicular failure, cryptorchidism, varicocele, testicular trauma, hydrocele, mumps, testicular dysgenesis syndrome, an endocrine disorder, a thyroid disorder, diabetes mellitus, a hypothalamic disorder, hyperprolactinemia, hypopituitarism and hypogonadism, or a subject that has reduced fertility as the result of alcohol abuse, drug abuse, or smoking.

These methods are well suited for autologous transplantation, wherein the GPR125-positive spermatogonial stem or progenitor cells are administered to the same subject from which the tissue sample was obtained. Such autologous transplantation methods are particularly useful for subjects in need of chemotherapy or radiation therapy, where the tissues samples may be removed from the subject before therapy, and the enriched and expanded GPR125-positive spermatogonial stem or progenitor cells may be administered to the subject after therapy.

One of skill in the art can readily perform such methods by preparing a therapeutic composition containing GPR125-positive spermatogonial stem cells, as described above, and administering the therapeutic composition to a suitable subject, such as a human patient, using the administration methods of described below.

The present invention encompasses methods of treatment performed by administering stem or progenitor cells, and methods of treatment performed by administering differentiated cells, or partially differentiated or committed cells, that have been derived from GPR125-positive stem or progenitor cells in vitro. For example, in the case of spermatogonial stem and progenitor cells, the present invention encompasses methods of treatment performed by administering differentiated spermatogonial cells derived in vitro from GPR125-positive stem or progenitor cells.

Methods of Treatment Using Differentiated Cells Derived from GPR125-Positive Stem or Progenitor Cells.

The present invention also provides methods of treatment comprising administration of differentiated cells derived from GPR125-positive stem or progenitor cells to subjects. In the case of GPR125-positive SPCS, differentiated spermatogonial cells derived therefrom by be administered to subjects in need thereof. In the case of GPR125-positive skin stem cells, differentiated skin cells derived therefrom may be administered to subjects in need thereof. In the case of GPR125-positive gut cells, differentiated gut cells derived therefrom by be administered to subjects in need thereof. In the case of GPR125-positive retinal cells, differentiated retinal cells derived therefrom may be administered to subjects in need thereof. Importantly, in the case of GPR125-positive MASCs, differentiated cells of multiple different types may be derived therefrom and may be be administered to subjects in need of those particular cell types. For example, GPR125-positive MASCs may be differentiated into endodermal cells, ectodermal cells, mesodermal cells, mucin-positive endoderm, GFAP+ neuroectoderm, chondrocytic cells, osteogenic cells, chondrogenic cells, GCNA+ gonad cells, myoid cells, vascular endothelial cells (capable of forming functional blood vessels), cardiac cells, neurons, gut cells, skin cells, and the like, and the differentiated cells may be administered to subjects in need thereof.

In preferred embodiments, these methods comprise obtaining a tissue sample, enriching and expanding the GPR125-positive stem or progenitor cells from the tissue sample in vitro, differentiating the GPR125-positive stem or progenitor cells in vitro, and then administering the differentiated cells to the subject. One of skill in the art can readily perform such methods by preparing a therapeutic composition containing the differentiated cells derived from the GPR125-positive stem or progenitor cells, as described above, and administering the therapeutic composition to a suitable subject, such as a human patient, using the administration methods of described below.

In preferred embodiments, the present invention provides methods for autologous transplantation, wherein a tissue sample is obtained from a subject, the GPR125-positive stem or progenitor cells from the tissue sample are enriched and expanded in vitro, for example using the methods described above, the GPR125-positive stem or progenitor cells differentiated into the desired cell type in vitro, and the differentiated cells are then administered to the same subject from which the tissue sample was obtained, for example using the administration methods described below.

Methods of treatment using differentiated cells derived from GPR125-positive stem or progenitor cells may be particularly useful when the subject is suffering from, or is at risk of developing, a disease, disorder, or condition associated with a lack of, or defect in, cells of that type. For example, methods of treatment using endothelial cells derived from GPR125-positive stem cells may be particularly useful when the subject is suffering from, or is at risk of developing, an ischemic condition or other condition affecting the vasculature. Similarly, methods of treatment using neuronal cells derived from GPR125-positive stem or progenitor cells may be particularly useful when the subject is suffering from, or is at risk of developing, a disease, disorder, or condition affecting the nervous system (including the retina) such as spinal cord injury, traumatic brain injury, a neural tumor, a neurodegenerative disease, Parkinson's disease, Alzheimer's disease, Lewy body dementia, Creutzfeldt-Jakob disease, Huntington disease, multiple sclerosis, traumatic retinal injury, retinopathy, retinoblastoma, a retinal degenerative disease or macular degeneration. Each type of differentiated cell that can be derived from GPR125-positive stem or progenitor cells may be useful for treating subjects suffering from, or at risk of developing, a condition associated with a lack of that cell type or a defect of that cell type.

One of skill in the art can readily perform such treatment methods by preparing a therapeutic composition containing differentiated cells derived from GPR125-positive stem cells, as described above, and administering the therapeutic composition to a suitable subject, such as a human patient, using the administration methods described below.

### Administration of GPR125-Positive Stem or Progenitor Cells, or Differentiated Cells Derived Therefrom, to Subjects

Several of the embodiments of the invention involve administration of GPR125-positive stem or progenitor cells, or differentiated cells derived therefrom, to subjects. The cells may be administered to subjects using any suitable means known in the art. For example, the cells may be administered by injection or infusion into the blood stream at a location peripheral to the site where the cells are needed, or by injection or infusion into the blood stream in the vicinity of the region where the cells are needed, or by direct infusion or injection into tissue, either at the site where the cells are needed, or in the vicinity of the site where the cells are needed, or at a peripheral location. In the case of GPR125-positive spermatogonial stem cells, it is preferred that the cells are administered directly into the testis. In the case of GPR125-positive skin stem cells, it is preferred that the cells are administered directly into the skin, such as by intradermal injection. In the case of GPR125-positive intestinal stem cells, it is preferred that the cells are administered directly to the region of the intestinal tract where they are needed, such as the colon, bowel, small intestine, large intestine, stomach or esophagus. In the case of GPR125-positive neural stem cells, it is preferred that the cells are administered directly to the region of the nervous system where they are needed, such as a specific brain region, a region of the spinal cord, a particular region of the peripheral nervous system, or the retina. Where differentiated cells are to be used, again it is preferred that the cells be administered locally to the site where they will be needed. For example, in the case of differentiated neuronal cells, it is it preferred that the cells are administered directly to the region of the nervous system where they are needed. In the case of differentiated cardiac cells, it is preferred that the cells are administered to the heart. The cells may be administered in a single dose, or in multiple doses. The skilled artisan will be able to select a suitable method of administration according to the desired use.

### Methods of Drug Targeting

In certain embodiments, the present invention provides a method of targeting a therapeutic agent to a stem or progenitor cell in a subject by conjugating a therapeutic agent to an agent that binds to GPR125 and administering the conjugated agent to the subject. Such methods can be used to target therapeutic agents, such as drugs, to any GPR125-positive cells, such as GPR125-positive spermatogonial stem or progenitor cells, skin stem or progenitor cells, intestinal stem or progenitor cells, neural stem or progenitor cells, or cancer stem cells. In preferred embodiments, the GPR125-binding agent binds to the extracellular domain of GPR125.

For example, therapeutic agents that may be targeted to GPR125-positive cells include, but are not limited to, cytotoxic drugs, other toxins and radionuclides. Such conjugates would be particularly useful in where the GPR125-positive cells are GPR125-positive cancer cells, or other GPR125-positive cells that are over-proliferative. In preferred embodiments, the therapeutic agents are conjugated to an antibody that binds to GPR125, preferably an antibody that binds to the extracellular domain of GPR125, and preferably a humanized monoclonal antibody. Methods of conjugating therapeutic agents to antibodies are known in the art, and any such method can be used.

### GPR125 and the GPR125 Ligand as a Drug Target

It is possible that GPR125, and its ligand, may be functionally involved in stem cell processes such as maintaining a de-differentiated state, maintaining proliferation, and the like. Agents that modulate the function of GPR125 or its putative ligand may therefore be useful. Thus, in one aspect, the present invention is directed to agents that modulate the function of GPR125 or its ligand(s) and to methods of identifying such agents. Such agents may be useful, inter alia, as anti-tumor drugs, or as agents for maintaining stem cells in culture, or as agents for facilitating differentiation of stem cells into differentiated cells types.

### Cancer Stem Cells

The present invention provides methods involving cancer cells. These methods are based on the discovery that GPR125 may be a marker of cancer stem cells. All of the embodiments described herein can be applied to GPR125-positive cancer cells. Thus, in one embodiment, the present invention provides a method of detecting a cancer stem cell comprising contacting a tissue, tissue sample or cell population with an agent that binds to GPR125 and determining whether the agent has bound to the tissue, tissue sample or cell population, wherein binding of agent indicates the presence of a cancer stem cell and an absence of binding indicates an absence of cancer stem cells. In another embodiment, the invention provides a method of detecting a tumor comprising contacting a tissue, tissue sample or cell population with an agent that binds to GPR125 and determining whether the agent has bound to the tissue, tissue sample or cell population, wherein binding of the agent indicates the presence of tumor cells and an absence of binding indicates an absence of tumor cells. The present invention also provides methods for determining whether a subject is likely to develop cancer, by determining whether a tissue, tissue sample or cell population from the subject contains one or more GPR125-positive cancer stem cells or tumor cells. It is believed that the presence of such cells may provide an early prognostic marker, and thus be useful for detecting tumors, or subjects likely to develop tumors, at an early stage, allowing appropriate preventative or therapeutic regimens to be initiated early.

The drug targeting methods described above, are particularly well suited to use with GPR125-positive cancer cells. Such methods can be used to target chemotherapeutic drugs, radionuclide drugs, or other toxic agents to GPR125-positive cancer stem cells, thereby killing the GPR125-positive cancer stem cells but not the surrounding non-cancerous tissue.

These and other embodiments of the invention are further described in the following non-limiting examples.

### EXAMPLES

### Example 1

### GPR125 as a Marker of Stem and Progenitor Cells

The numbers in superscript below refer to the numbered references provided in the reference list that immediately follows this example.

Adult mammalian testis is a source of pluripotent stem cells¹. However, the lack of specific surface markers has hampered identification and tracking of the unrecognized subset of germ cells that gives rise to multipotent cells². While embryonic-like cells can be derived from adult testis cultures after only several weeks in vitro¹, it is not known whether adult self-renewing spermatogonia in long-term culture can generate such stem cells as well. The present Example shows that highly proliferative adult spermatogonial progenitor cells (“SPCs”—also referred to as “SPs” or “SSCs”) can be efficiently obtained by cultivation on mitotically-inactivated testicular feeders containing CD34⁺ stromal cells. SPCs exhibit testicular repopulating activity in vivo and maintain the ability to give rise in long-term culture to multipotent adult spermatogonial derived stem cells (“MASCs”). Furthermore, both SPCs and MASCs express GPR125, an orphan adhesion-type G-protein coupled receptor. In knock-in mice bearing a GPR125-β-galactosidase (β-gal) fusion protein under control of the native GPR125 promoter (GPR125βgal), expression in the testis was detected exclusively in spermatogonia and not in differentiated germ cells. Primary GPR125βgal SPC (GSPC) lines retained GPR125 expression, underwent clonal expansion, maintained the phenotypic repertoire of germline stem cells, and reconstituted spermatogenesis in busulfan-treated mice. Long-term cultures of GPR125⁺SPCs also converted into GPR125⁺ MASC colonies. GPR125⁺ MASCs generated derivatives of the three germ layers and contributed to chimeric embryos, with concomitant down-regulation of GPR125 during differentiation into GPR125^{negative} progeny. MASCs also differentiated into contractile cardiac tissue in vitro and formed functional blood vessels in vivo. Molecular bookmarking by GPR125 in the adult mouse and ultimately human testis could enrich for a population of SPCs for derivation of GPR125⁺ MASCs that may be employed for genetic manipulation, tissue regeneration, and revascularization of ischemic organs.

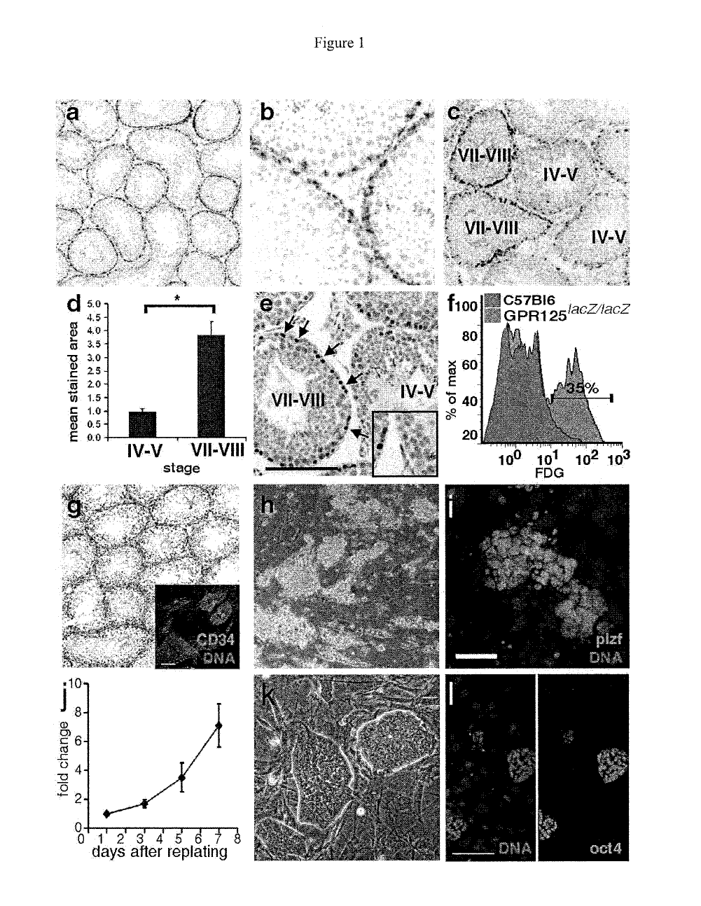

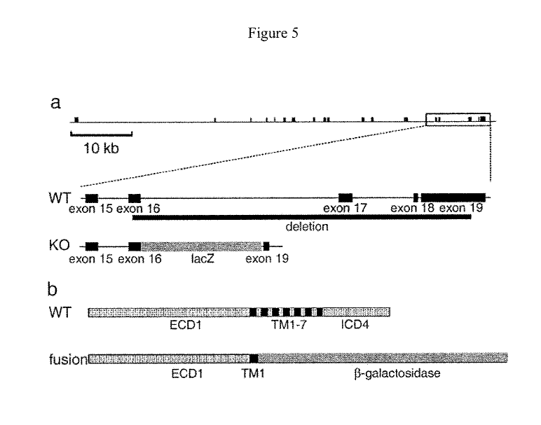

The genetic and phenotypic repertoire of the specific subset of spermatogonial cells that converts into multipotent adult cells is poorly defined. In the present example, it is shown that a potential stem and progenitor cell surface marker (GPR125) expressed on the adult testis. This was discovered in the course of evaluating a large series of mouse knockouts³. The endogenous GPR125 locus was altered by joining the N-terminal putative extracellular and first transmembrane domains to β-galactosidase (FIG. 5). Homozygous mice were grossly normal and fertile. Histochemical examination of the post-natal testis by β-galactosidase substrate X-gal revealed that GPR125 expression was restricted to the seminiferous tubules and was confined within the first layer of cells adjacent to the basement membrane of the peritubular cells (FIG. 1a-c). Immunohistochemistry revealed GPR125 expression only in spermatogonia (FIG. 1e).

As spermatogenesis proceeds along the length of the seminiferous tubule, characteristic sets of differentiating cell types are seen together in a given cross-section, allowing such cross-sections to be categorized into twelve stages⁴. Expression of GPR125 was greatest at later stages (i.e., VII-VIII) with a nadir in earlier stages (i.e., IV-V) as analyzed either by promoter activity (X-gal) or by immunostaining (in wild type mice; FIG. 1c-e). To quantitate expression of GPR125βgal in the GPR125^{lacZ/lacZ} spermatogonia, staining was performed with fluorescein di-D-galactopyranoside (FDG), followed by flow cytometry. Freshly dissociated adult GPR125^{lacZ/lacZ} seminiferous tubules yielded ˜35% βgal⁺ cells (FIG. 1f). The high yield of βgal⁺GPR125⁺ cells may be a result of our preparation of testicular tissue, in which contaminating interstitial somatic cells and spermatids are lost during washing steps, combined with the high sensitivity of the FDG assay⁵.

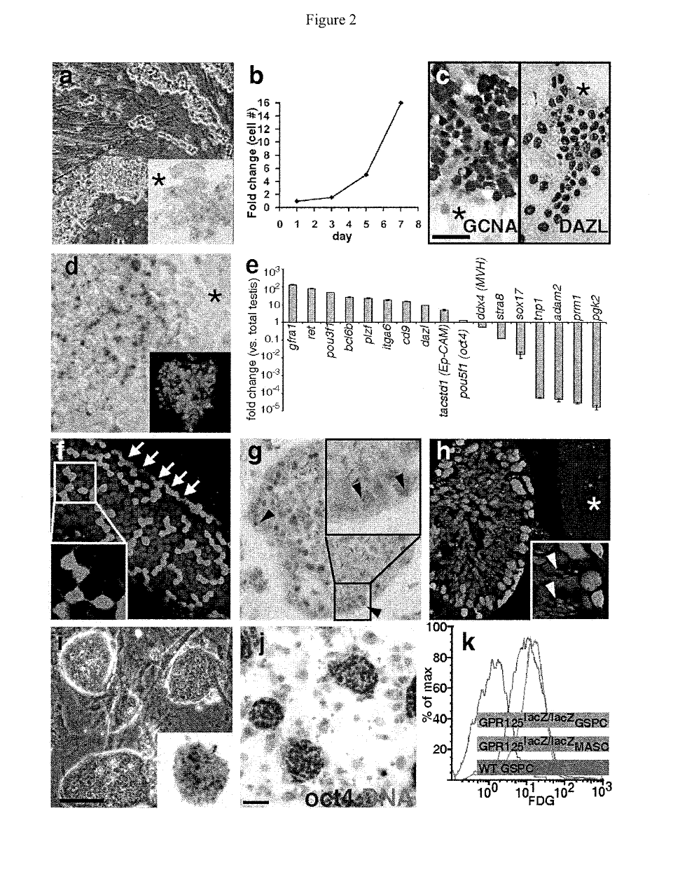

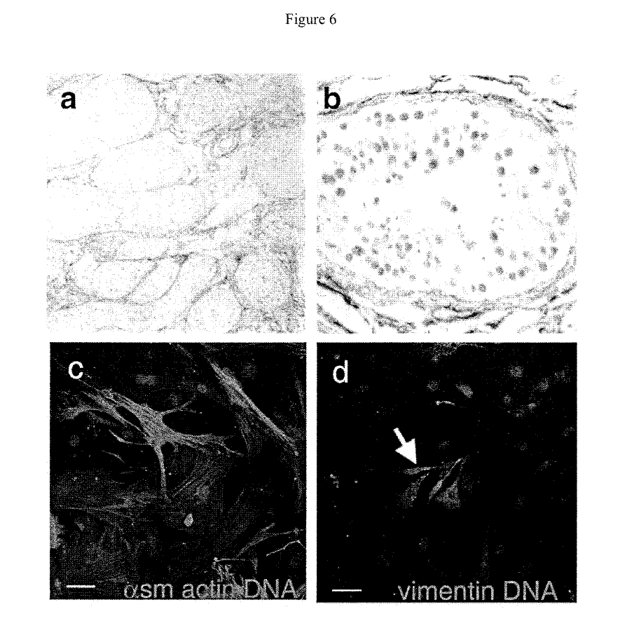

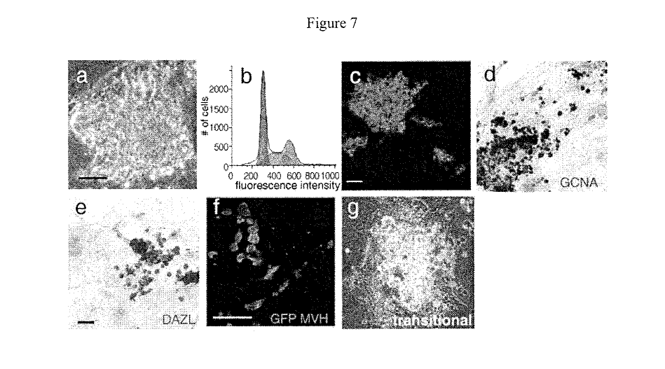

To determine whether βgal⁺ GPR125⁺ cells represent self-renewing spermatogonial cells with the capacity to generate MASCs, we sought to recapitulate in vitro the native niche that supports efficient self-renewal of these cells. It was discovered that the βgal⁺GPR125⁺ cells reside in close proximity of the CD34⁺ peritubular cells⁶, suggesting that interaction of these two cell types may be essential for expansion of the GPR125⁺SPCs (FIG. 1g and FIG. 6a-b) To culture GPR125⁺ cells, primary mitotically-inactivated adult mouse testicular stromal cells were established containing CD34⁺ putative peritubular cells (CD34⁺ mTS), since initial attempts using mouse embryo fibroblasts (MEFs) were unsuccessful. Amongst the CD34⁺ stromal cells were also α-smooth muscle actin⁺ and vimentin⁺ cells that together supported derivation and long-term proliferation of adult SPCs from mouse testes of various ages (up to 1 year) and genetic backgrounds in >90% of attempts (FIG. 1g, inset and FIG. 6c-d). The adult spermatogonial cultures displayed heterogeneous colony size, with frequent formation of massive proliferating colonies, exponential overall growth, and ˜30% of cells in S-phase (FIG. 1h,j and FIG. 7a-c). Adult SPC lines were also derived from mice displaying green fluorescence in all tissues' and were serially passaged six times in typical fashion on CD34⁺ mTS, revealing expansion of SPCs and near total (>99%) depletion of any green fluorescent protein (GFP)-positive cells outside of the characteristic spermatogonial-stem cell-like colonies, suggesting loss of the non-germline contaminants (FIG. 7c). The SPC lines expressed typical mouse germ lineage markers, including germ cell nuclear antigen (GCNA), DAZL, and MVH (FIG. 7d-f)⁸⁻¹⁰. Notably, the colonies expressed the well-characterized marker plzf, which identifies undifferentiated spermatogonia (FIG. 1i)^{11,12} Evidence of bona fide stem cell activity within the SPC pool (cultured for more than one year) was revealed by their ability to participate in reconstitution of spermatogenesis in busulfan-treated host mice (see FIG. 2)¹³.

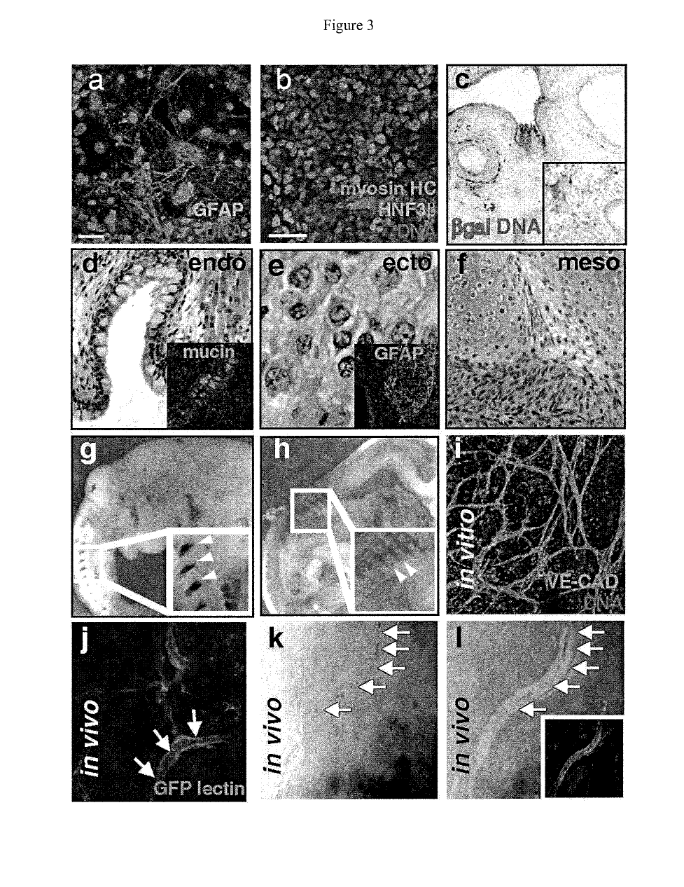

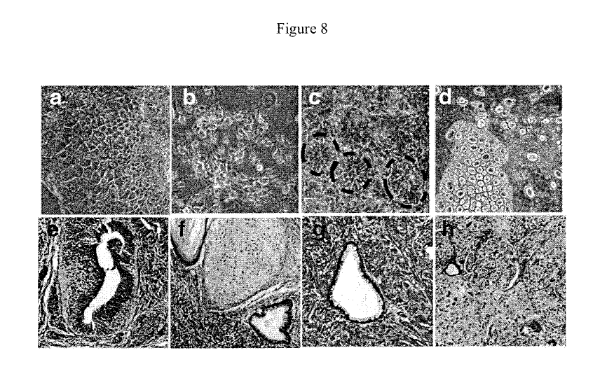

Prior studies have found that embryonic stem cell (ESC)-like cells arose either from neonatal testicular cells through spontaneous conversion in the presence of glial cell line derived neurotrophic factor (GDNF) and leukemia inhibitory factor (LIF) on mouse embryo fibroblasts (MEFs)¹⁴ or in adult SSC cultures maintained in the absence of GDNF within four weeks after the initiation of spermatogonial colonies¹. We found that long-term culture of adult SPCs generated distinct colonies of MASCs from cells that were originally cultured on the CD34⁺mTS feeder layers for more than three months (FIG. 1k-l). The emergence of MASC colonies was heralded by a distinct morphologic change in a subset of SPC colonies (FIG. 7g). Putative MASC colonies, resembling ESCs, were mechanically transferred off CD34⁺mTS onto MEFs for MASC expansion in the undifferentiated state (FIG. 1k)¹⁵. While the pluripotency marker oct4 protein was undetectable in SPCs (data not shown), unequivocal oct4 expression was found in the nuclei of MASCs that were expanded (15 passages before cryopreservation) on MEFs (FIG. 1L) and that were capable of differentiation into multiple lineages in vitro, including rhythmically contractile cardiogenic tissue (FIG. 8a-d). MASCs gave rise to teratomas (9/9 attempts) when injected subcutaneously in NOD-SCID mice (FIG. 8e-h). The expression of βgal in both ROSA26βgal¹⁶ MASCs and the resultant teratomas, excluded the possibility of a multipotent mesenchymal cell originating from the wild type, mitomycin-C inactivated feeders. Furthermore, MASCs cloned from single cells were similarly competent to form tri-lineage teratomas and contribute to chimeric embryos upon blastocyst injection (see FIG. 3).

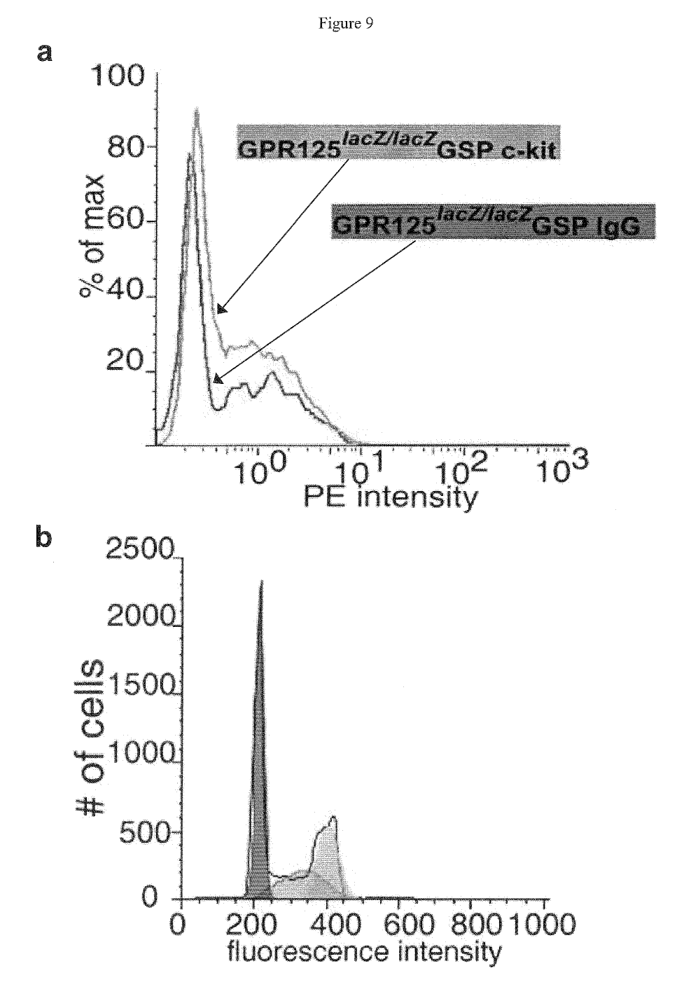

To determine whether GPR125 is expressed on SPCs, GPR125^{+/lacZ} and GPR125^{lacZ/lacZ} testes were used to derive SPC lines propagated on CD34⁺mTS. Refractile, cobblestone colonies reminiscent of SSCs appeared within one week, and large proliferative colonies were seen within 3-4 weeks, exhibiting exponential clonal growth, and culture wells could be de-populated with complete re-growth of colonies (FIG. 2a-b). Maintenance of the germ cell phenotype was confirmed by immunohistochemistry for GCNA and DAZL (FIG. 2c)¹⁷, but c-Kit was absent by flow cytometry (FIG. 9a). Strikingly, GPR125^{lacZ/lacZ} SPCs maintained GPR125 expression after multiple passages in vitro (FIG. 2a, inset) and are hereafter referred to as GPR125⁺ SPCs (GSPCs). To determine the frequency of repopulating cells, limiting dilution analysis was performed using GFP-labeled GPR125^{lacZ/lacZ} GSPCs on cells that were cultured beyond nine months, revealing 0.23 (95% confidence interval: 0.19-0.27) colony forming units (CFU) per cell or 1 CFU for every 4-5 GSPCs (FIG. 2d). All emerging colonies derived from the testes of GPR125^{lacZ/lacZ} mice expressed lacZ, suggesting that the GSPCs are clonagenic (FIG. 2d).

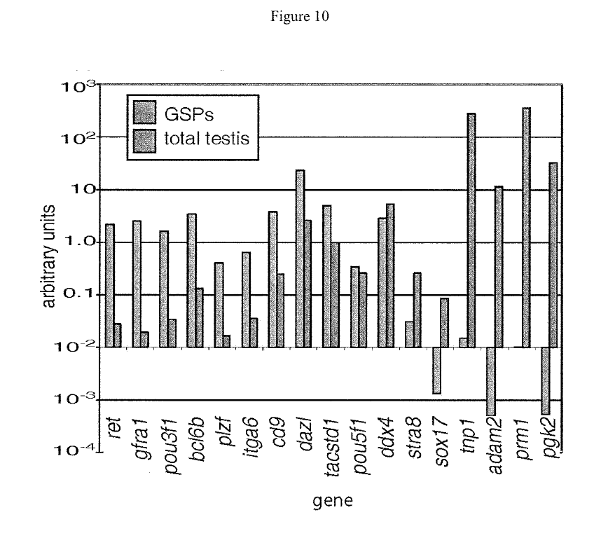

The molecular identity of GPR125^{lacZ/lacZ} GSPCs in long-term culture was confirmed by quantitative PCR (FIG. 2e FIG. 10). Among the transcripts expressed in GPR125^{lacZ/lacZ} GSPC cultures were germ cell-specific genes, including DAZL and MVH^{10,17}. To rule out spontaneous spermatogenic differentiation of the cultured GSPCs¹⁸, transcripts characteristic of differentiated germs cells were surveyed, and diminished or absent levels for transcripts, such as sox17, transitional protein-1, fertilin beta (adam2), protamine-1, and phosphoglycerate kinase 2¹⁹, were noted. These data suggested that repopulating GSPCs were of germ cell origin but remained undifferentiated. Even after in vitro propagation for over one year, GPR125^{lacZ/lacZ} GSPCs revealed a transcriptional profile highly reminiscent of spermatogonial stem cells (FIG. 2e and FIG. 10). Various cell surface markers used for isolation of SSCs were increased in GSPCs: α6 integrin (˜18-fold), Ep-CAM (˜5-fold), CD9 (˜15-fold), and GFRa1 (˜128-fold)²⁰⁻²². Similarly, genes utilized for their preferential promoter activity in undifferentiated cells were detectable albeit at lower levels in the GPR125⁺ cells, including stra8 and oct4. Therefore, this culture technique yields undifferentiated spermatogonia, which like spermatogonia in vivo, express GPR125.

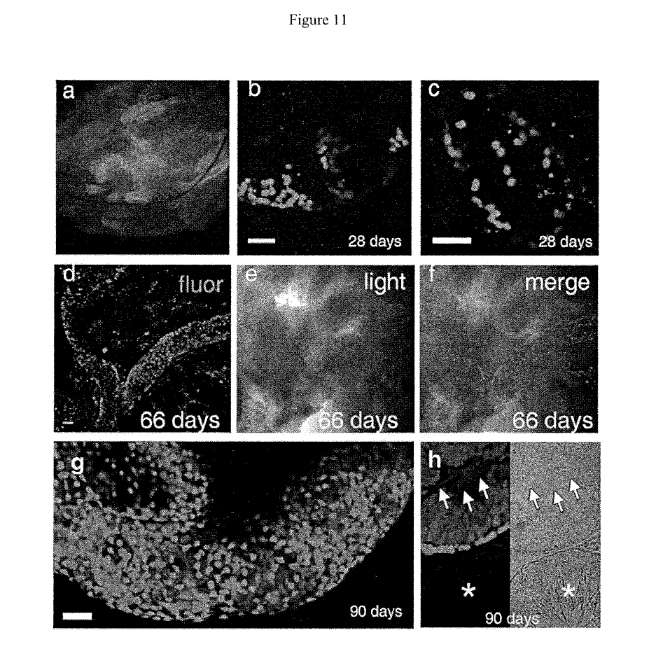

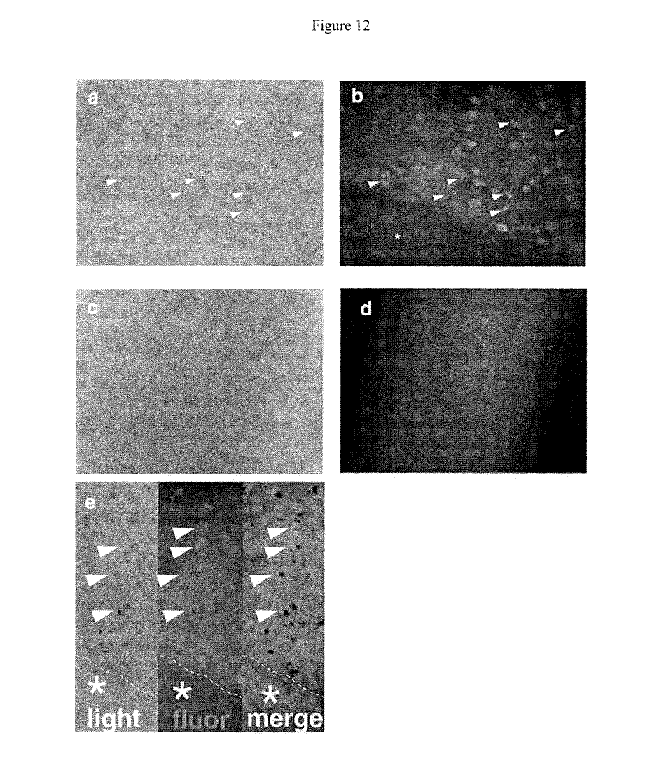

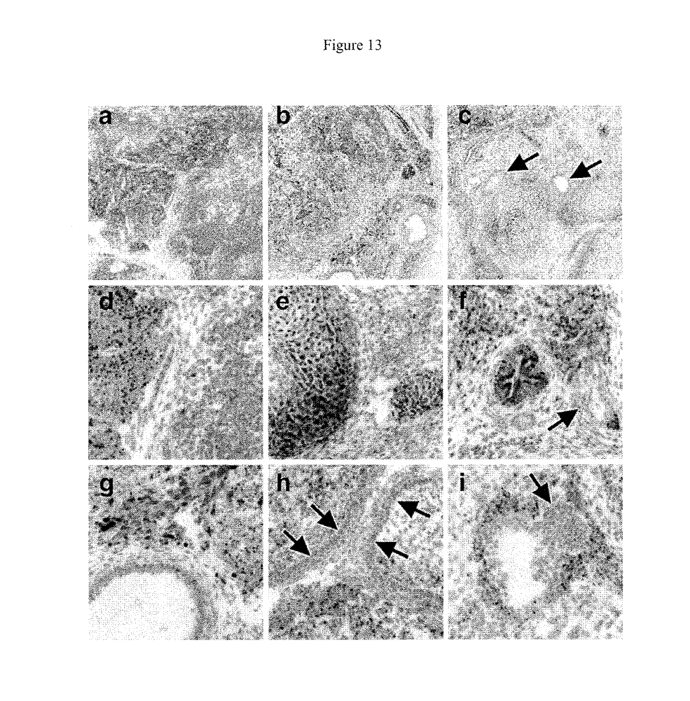

To interrogate the repopulating potential of GSPCs in vivo, the capacity of GFP-labeled GPR125^{lacZ/lacZ} GSPCs to restore spermatogenesis within busulfan-treated C57B16 host mouse testes was evaluated¹³. Within 2-3 months after transplantation, robust GFP⁺ GPR125^{lacZ/lacZ} germ cell colonies were detectable within the host seminiferous tubules (FIG. 2f and FIG. 11a). These colonies contained populations of GFP⁺ cells along the basement membrane, exhibiting typical spermatogonial morphology, and smaller round GFP⁺ cells located more centrally to tubular lumen (FIG. 2f and FIG. 11b-g). X-gal staining confirmed co-expression of GPR125 (lacZ⁺) in a small subset of the GFP-labeled, transplanted cells, along the basement membrane (FIG. 2g and FIG. 12a-e), recapitulating the spatial expression pattern in the GPR125βgal testes (see FIG. 1). Importantly, GFP⁺ spermatids were seen in donor-colonized tubules but not in adjacent tubules containing residual, host-derived spermatogenesis, confirming the presence of true stem cell activity within the long-term GPR125^{lacZ/lacZ} GSPC cultures (FIG. 2h and FIG. 13h). PCR for GFP detected donor-derived sperm in the epididymis draining the transplanted testis but not in negative controls (data not shown).

The origin of multipotent stem cells in the adult testis is not clear²³. Therefore, it was sought to formally prove that GSPCs could indeed generate multipotent cells, even after long-term expansion in vitro. The spontaneous emergence of MASCs was observed in the GPR125^{lacZ/lacZ} cultures that were initially propagated for more than 3 months. These GPR125^{lacZ/lacZ} MASCs had a high nuclear-to-cytoplasmic ratio, formed refractile colonies and could be split ˜1:8 every 2-3 days (FIG. 2i; passaged >30 times before cryopreservation). The majority of cells had a normal karyotype, and no evidence of clonal cytogenetic abnormalities was found for either GPR125^{lacZ/lacZ} MASCs or Rosa26βgal MASCs (data not shown). Notably, the majority of cells within the colonies were highly positive for GPR125 expression and also uniformly immuno-positive for oct4 within the nucleus (FIG. 2j). FDG labeling revealed more than 99% of both GPR125^{lacZ/lacZ} GSPCs and MASCs to be GPR125⁺ by β-galactosidase activity (FIG. 2k), suggesting that GPR125 is associated more universally with the stem and progenitor cell phenotype.

The multipotency of these GPR125^{lacZ/lacZ} MASCs was assessed first by formation and differentiation of embryoid bodies (EBs) in vitro²⁴. Within seven days after re-plating, EBs exhibited a distinct pattern of GPR125 expression, with distinct borders between GPR125⁺ and GPR125^{negative} areas. The resultant colonies contained HNF3β⁺ cells derived from endoderm or ectoderm, cytokeratin⁺ or GFAP⁺ cells derived from ectoderm, and brachyury⁺ or skeletal muscle myosin⁺ derived from mesoderm cells (FIG. 3a-b).

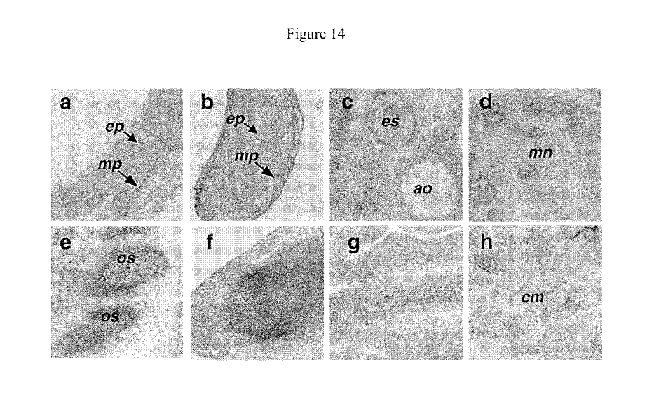

When GPR125^{lacZ/lacZ} MASCs were implanted subcutaneously in NOD-SCID mice, the resultant teratomas (14/14 attempts) similarly exhibited GPR125 expression in a lineage-specific manner, implying loss of GPR125 in certain differentiated cell types (FIG. 3c and FIG. 13). In fact, these teratomas were reminiscent of GPR125βgal embryos, in which GPR125 expression is present in most but not all tissues and subsequently lost over time (see FIG. 3h, FIG. 14). Lineage analysis of MASC teratomas demonstrated morphologic and immunologic evidence for tissue derivatives of all three germ layers, including mucin-positive endoderm, GFAP⁺ neuroectoderm, and mesodermal chondrocytic, myoid, and vascular cells (FIG. 3d-f).

The ability to form chimeric animals has been used to demonstrate multipotency of germ cell derivatives². We therefore performed blastocyst injections with cloned GPR125^{lacZ/lacZ} MASCs and found 8 (22%) chimeric embryos out of 37 evaluated. Importantly, the expression pattern of GPR125 in the C57B16 (host)/GPR125^{lacZ/lacZ} (donor) chimeric embryos partially recapitulated what was seen in heterozygous knock-in GPR125^{+/lacZ} embryos, with prominent signal in developing ossification centers (FIG. 3g-h and FIG. 14e-f). In addition, βgal⁺ cells were also detected in the chimeric gut and other tissues that are known FIG. 14). These data indicate that generation of GPR125⁺ MASCs from GSPCs results in the maintenance of the expected global expression pattern of GPR125 gene. As such, lineage-specific derivatives of MASCs may have the essential genetic and epigenetic critical for autologous organ regeneration

To this end, the ability of MASCs to differentiate into endothelial cells was examined. An extensive network of vessel-like, lumen-containing VE-cadherin⁺ structures were formed in vitro from MASC embryoid bodies after 22 days of differentiation (FIG. 3i and data not shown). To determine whether GPR125⁺ MASCs could differentiate into functional vessels in vivo, GPR125⁺ MASCs were transduced with a lentiviral vector expressing GFP under control of the promoter for the endothelial-specific marker VE-cadherin²⁵. Teratomas formed in NOD-SCID mice from such transduced MASCs contained donor-derived GFP⁺ blood vessels, continuous with the host circulation, as evidenced by perfusion-based staining and the presence of red blood cells within the vessels (FIG. 3j-1).

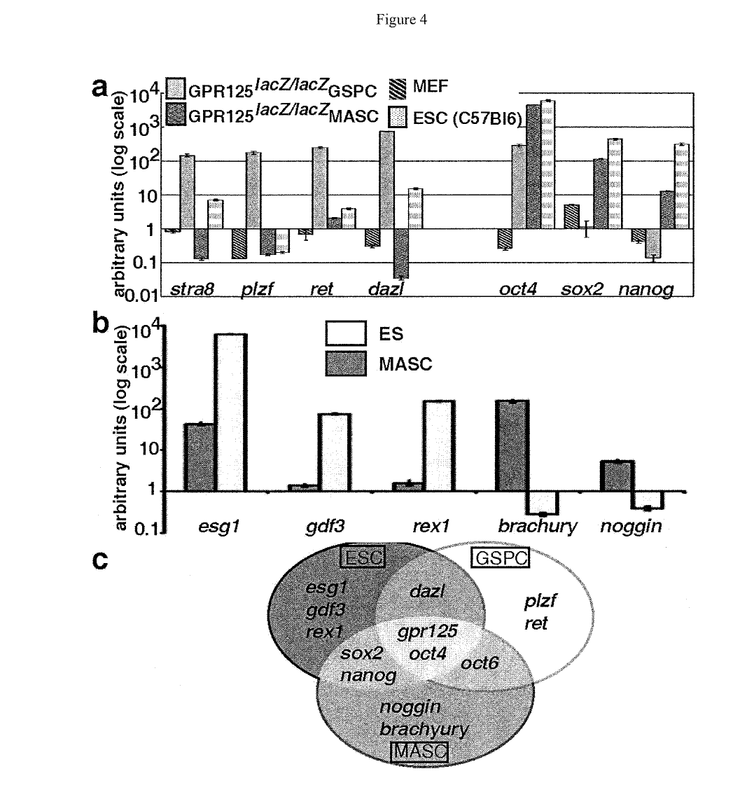

It was asked next whether MASCs utilize the same molecular machinery for multipotency as ESCs. Expression analysis of GPR125^{lacZ/lacZ} MASCs compared to mouse ESCs, GSPCs, or MEFs revealed high levels of oct4, nanog, and sox2 in both MASCs and ES cells (FIG. 4a) Minimal expression of typical SSC markers, including plzf, ret, and stra8, was seen in MASCs, which, as expected, were high in GPR125^{lacZ/lacZ} GSPCs. Unexpectedly, certain key germ lineage transcripts (e.g., DAZL) were nearly absent in MASCs, as were some canonical mouse ESC transcripts (e.g., gdf3, esg1, and rex1; FIG. 4b). The differences in expression of these genes and others (e.g., noggin and brachyury) suggest that MASCs constitute a distinct stem cell type from that reported by Guan et al¹.

The present study has identified, for the first time, GPR125 as a surface marker for self-renewing, clonagenic, cKit^{negative}plzf⁺ spermatogonial progenitor cells (GSPCs), with the capacity for both repopulating the testis and generating GPR125⁺ MASCs. Recent evidence indicates that spermatogonial progenitor cells can manifest stem cell activity²⁶. This suggests that GPR125⁺cKit^{negative}plzf⁺DAZL⁺ GSPCs may not only be endowed with spermatogonial stem activity but also perform as undifferentiated spermatogonial cells that can convert into GPR125⁺cKit⁺Plzf^{negative}Dazl^{negative}Oct4⁺ MASCs. These data pinpoint GPR125⁺spermatogonial cells as the cellular ancestors of MASCs. Differentiation of GPR125⁺ MASCs into GPR125^{negative} tissues qualifies GPR125 expression as a useful marker for tracking differentiation and lineage-specification of stem and progenitor cells.

The precise molecular and cellular pathways governing the emergence of MASC colonies remain unclear. Although MASCs and ESCs have identical morphological characteristics and are both multipotent, capable of giving rise to teratomas and chimeric animals, there are major differences at the transcriptional level that distinguish these two cell types (FIG. 4c). Notably, unlike the ES-like cells derived from stra-8⁺ SSCs¹, GPR125⁺ MASCs lack the molecular signature of ES cells but mimic other multipotent adult stem cells, such as multipotent adult progenitor cells (MAPCs)²⁷. The data presented herein, therefore, implies that multipotency may be driven by multiple unique sets of signals, even in the absence of gene products typically associated with sternness (e.g., gdf3, esg1, and rex1). Also, in contrast to a prior report', the maintenance of long-term cultures of GPR125⁺ SPCs was dependent on GDNF and was therefore necessary for the subsequent emergence of MASCs. Therefore, culture conditions may influence the ultimate multipotent phenotype. GPR125 expression in undifferentiated cells and early progenitors and its subsequent down-regulation upon terminal differentiation raises the intriguing possibility of exploiting surface expression of GPR125 to isolate human SSCs and SPCs. Recent data demonstrated the in vitro differentiation of endothelium from multipotent cells derived from the neonatal testis²⁸. The present study extended these observations by showing that GPR125⁺ MASCs can generate functional vascular cells in vivo. Taken together, these data suggest that GPR125⁺ MASCs could be used therapeutically for the generation of functional autologous vessels for revascularization.

TABLE 1
Table 1 - GSPC lines created to date using
primary testicular feeder cells (MTS).

| | Age of | Approximate | |
|---|---|---|---|
| | donor | time in culture | |
| Mouse strain | (weeks) | (months) | Passage |
| UBC-GFP | 28 | >12 | 11 |
| UBC-GFP | 1 | 7.0 | 9 |
| FVB | 2 | 6.0 | 8 |
| GPR125^{+/+} | 48 | 5.0 | 3 |
| GPR125^{+/lacZ} | 3 | 6.0 | 4 |
| GPR125^{lacZ/lacZ} | 3 | 11.5 | 14 |
| ROSA26-lacZ | 12 | 6 | 6 |
| Sl^{d}/Sl^{d} | 28 | 2.5 | 2 |
| C57Bl6/129S | 1 | 3.5 | 7 |
| GPR125^{lacZ/lacZ} | 16 | 6.5 | 7 |

GSPC lines were derived as described in the below Materials and Methods section of this Example.

TABLE 2
Table 2 - Tissues populated by
GPR125^{lacZ/lacZ} MASCs in chimeric animals.

| Tissue | Chimerism |
|---|---|
| Gut | + |
| Skin | + |
| Ossification | + |
| centers | |
| Lung | + |
| Heart | + |
| Brain | − |
| Liver | − |

Clones of red fluorescent GPR125^{lacZ/lacZ} MASCs that had been previously labeled in vitro with mCherry driven by the PGK promoter were used to create chimeric animals by injection of C57B16 blastocysts at embryonic day 3.5 (E3.5) and assessed at E13.5 to P0, as described in the below Materials and Methods section of this Example. The presence of chimerism in different tissues was assessed by X-gal staining (light microscopy) or red fluorescence (confocal microscopy).

### Materials and Methods

### SSC, MASC, and Feeder Cell Culture

C57B16 mice aged 4-12 weeks served as donors for mixed primary testicular feeder cells, which were expanded following enzymatic digestion of the seminiferous tubules. Feeder cells were treated with mitomycin-C prior to use for stem cell culture. Mouse SPCs were obtained from enzymatically dissociated seminiferous tubules from mice aged 3 weeks to 8 months and were plated in StemPro®-34 (Invitrogen) with the modifications of Kanatsu-Shinohara et al²⁹. SPCs were serially passaged onto fresh mitomycin-C-treated feeders every 2 to 8 weeks. Morphologically atypical transitional colonies of SPC were mechanically removed from the plate after >2 weeks in culture and re-plated in the same medium or ES medium on mitomycin-C-inactivated MEF to obtain MASC lines.

### GPR125βgal Mice

VelociGene® technology was employed for production of GPR125^{lacZ/lacZ} mice as previously described³. Briefly, targeting vectors were generated using a bacterial artificial chromosome (BAC) and contained gpr125 in which the exons 16-19 were deleted and replaced in-frame with lacZ, as a reporter gene and neomycin as a selectable marker. Targeting vectors were electroporated into ES cells. Clones that were properly targeted were confirmed by the real-time PCR-based loss-of-native-allele assay³ using primers listed below. Chimeric mice were generated by blastocyst injection of ES cells and backcrossed to C57B16/J to produce heterozygote breeding pairs.

### Gene Targeting

The primers to identify the 5′ junction of the mutant GPR125 allele included forward primer ATGTTAGCTT-AAATGGACTGTC (SEQ ID NO: 3) and reverse (lacZ) GTCTGTCCTA-GCTTCCTCACTG (SEQ ID NO: 4), and for the 3′ junction, included forward primer (neo) TCATTCTCAGTATTGTTTTGCC (SEQ ID NO: 5) and reverse ATAGTAAATCCCAAAGCTCAC (SEQ ID NO: 6).

### Animals

Teratomas were generated by injecting 0.5-1×10⁶ cells in Matrigel™ subcutaneously into 8 week old NOD-SCID mice. C57B16 were donors for testicular stromal cultures. ROSA26-lacZ, UBC-GFP, FVB, Steel Dickie, and C57B16/129S mice also served as donors for GSPs. C57B16 mice served as hosts for spermatogonial stem cell transplantation. Mice were bred, manipulated, and sacrificed under the guidelines of the Institutional Animal Care and Use Committee.

### Histology and Immunostaining

Tissues were dissected from the mice and either snap-frozen in OCT (Tissue Tek) or fixed overnight in 4% paraformaldehyde (Alfa Aesar) in PBS at 4° C. for paraffin embedding. X-gal staining for detection of galactosidase activity was performed on cryosections using an overnight incubation with substrate (Calbiochem) at 37° C. per the manufacturer's directions. For immunohistochemistry, paraffin sections were rehydrated and heated in Antigen Retrieval Solution (Dako). Primary antibodies used in this study included two rabbit polyclonal antisera against GPR125 peptides (Genesis Biotech, Inc.), rat monoclonal anti-GCNA (courtesy of Dr. G. Enders), rat monoclonal anti-E-cadherin (R&D Systems), mouse anti-DAZL (Abeam), mouse anti-α smooth muscle actin (Dako), rat anti-mouse CD34 (Abeam), mouse anti-human CD34 (QBEND10), mouse anti-vimentin (Chemicon), mouse anti-oct4 (R&D Systems), rabbit anti-VASA (Abeam), rabbit anti-mouse CD31 (RDI) anti-mouse HNF3β (Santa Cruz), anti-mouse GFAP (Dako), and mouse anti-mucin SAC (clone 45M1, Lab Vision). Primary incubation of antibodies performed overnight. Monoclonal hamster anti-mouse plzf was generated using a peptide corresponding to the plzf hinge region as will be described elsewhere. For IHC, detection of primary antibodies was performed with biotinylated donkey anti-rabbit IgG (Jackson Laboratories) or biotinylated mouse anti-rat IgM (Zymed, Inc.). Biotinylated secondary antibody was followed by streptavidin-horseradish peroxidase and amino-ethyl carbazole (AEC, Biomeda Corp.). For IF, primary antibodies were detected with FITC-conjugated goat anti-hamster antibody (eBioscience), cy2- or cy3-conjugated non-cross reacting donkey anti-rabbit, rat, or mouse antibody, or with biotinylated donkey secondaries (Jackson Laboratories) followed by Alexa546- or Alexa488-conjugated streptavidin (Invitrogen) for additional amplification. Staining of cells in vitro was performed identically except that permeabilization was carried out with 0.2% Triton X-100/10% normal donkey serum/PBS for 30 minutes prior to incubation with certain primary antibodies. Counterstaining was performed with TOPRO1 (Invitrogen) (for IF), hematoxylin and eosin (Dako) for IHC, or nuclear fast red (Vector Laboratories) for X-gal staining. Color images of IHC or X-gal staining were captured using an Olympus microscope and contrast-enhanced uniformly for images within each experiment using Adobe Photoshop 7.0 (San Jose, Calif.). Immunofluorescent images were captured using the Zeiss LSM 510 Meta confocal microscope (Carl Zeiss, Inc.) and pseudo-colored after capture.

### Cell Culture

Primary mouse testicular stromal cells (mTS) were prepared from 4-12 wk old C57B16 mice as follows. Seminiferous tubules were collected from detunicated testes and minced. The tissue was washed and then enzymatically dissociated with agitation at 37° C. in a buffer containing 0.017% trypsin (Cellgro), 17 μM EDTA (Cellgro), 0.03% collagenase (Sigma-Aldrich), and DNAse I (100 μg/ml; Sigma-Aldrich). The resultant cell suspension (non-filtered) was collected, plated in flasks coated with gelatin in a 50:50 mixture of alpha modified Eagle's medium/StemPro®-34 (Invitrogen) supplemented with 20% FBS (Gibco) and expanded two to seven passages. Cells were then cryopreserved for future use or plated in flasks coated either with Matrigel™ (BD Biosciences) diluted 1:40 (for the first 1-2 passages of GSPS, to improve adherence of stroma to the plate) or gelatin (for subsequent passages) at 0.4-1.0×10⁶ cells per 35 mm dish and treated with mitomycin-C (10 μg/ml; Sigma-Aldrich) for 2-4 hours prior to use for stem cell culture. The population of cells in the mTS was heterogeneous as depicted in FIG. 6. Primary cultures of mouse spermatogonial stem cells were obtained as follows. Mice from 3 wks to 8 months of age of the indicated genotypes were sacrificed. Seminiferous tubules were collected from 1 to 2 de-tunicated testes and minced. The tissue was washed in 50 ml of PBS/1% BSA (Sigma-Aldrich), centrifuged at 30 g, and the pellet containing only large tissue fragments was enzymatically dissociated with agitation at 37° C. in a buffer (3 ml) containing trypsin, EDTA, 0.03% collagenase, and DNAse I (100 μg/ml). The resultant cell suspension was collected and either cryopreserved or plated on the feeder cells described above in spermatogonial stem cell medium containing StemPro®-34 (Invitrogen) and supplements as follows: D(+)glucose 33.3 mM (Sigma-Aldrich), BSA 0.50%, MEM vitamin solution 1×(Gibco), 3-estradiol 110 nM (Calbiochem), progesterone 190 nM (Calbiochem), fetal bovine serum 1%, penicillin (100 U/ml)/streptomycin (100 μg/ml)/amphotericin 0.2 μg/ml (Mediatech), transferrin 100 μg/ml (Sigma-Aldrich), insulin 25 μg/ml (Sigma-Aldrich), human GDNF 10 ng/ml (R&D Systems), ESGRO (mLIF) 1000 U/ml (Millipore), human bFGF 10 ng/ml (Biosource), non-essential amino acid solution 1×(Gibco), L-glutamine 2 mM (Mediatech), putrescine 60 μm (Research Organics), sodium selenite 30 nM (Sigma-Aldrich), pyruvic acid 340 μM (Sigma-Aldrich), d(L)-lactic acid 11 μM (Baker), β-mercaptoethanol 50 μM (Gibco), ascorbic acid 100 μM (EMD), D-biotin 10 μg/ml (Calbiochem), and mouse EGF 20 ng/ml (BD Biosciences). Cells were maintained at 37° C. in 5% CO₂. Cells were fed three times per week. Serial passaging was performed non-enzymatically by gentle trituration of colonies every 2-8 weeks, in order to progressively isolate GSPS from contaminating donor-derived stromal cells. Culture wells could be partially depopulated of GSPs by gentle trituration of loosely adherent colonies without disturbing the feeder cells, with subsequent re-growth of colonies in the same wells after addition of fresh medium. In this way, a given well of feeders could support GSPs proliferation for up to 8 weeks. Subsequently, wells were then trypsinized for either cryopreservation or further passaging on fresh feeders. For limiting dilution analysis, gelatin coated 96-well plates of feeders were prepared using an outgrowth cell line of the mTS that could be passaged continuously. Five 96-well plates of GSPs were prepared by serial doubling dilution. The location of rows calculated to contain single cells was confirmed by phase and fluorescent microscopy to confirm the presence of single cells per well. Plates were then maintained for 17 days at 37° C. and scored for the presence of single large colonies (greater than ˜50 cells). The rows in which ˜10 cells had been initially plated were employed for statistical analysis (n=60 wells) to obtain normally distributed data on the frequency of colony forming cells per total number of cells initially plated. To obtain MASC colonies, distinct clusters of GSPs with atypical, transitional morphology were identified by phase microscopy, mechanically separated from the plate using Pasteur pipettes, and replated on mitomycin-C-inactivated CF1 MEF (Chemicon) in the same GSP culture medium or ESC medium (see below). MASC were passaged with trypsinization every 2-4 days onto fresh inactivated MEF. C57B16 mouse ESCs were cultured using standard procedures. Mouse ESC culture medium consisted of KO-DMEM (GIBCO), 15% FBS, 1× non-essential amino acids, 1× penicillin/streptomycin antibiotic, 2 mM L-Glutamine, 55 μM β-mercaptoethanol, and leukemia inhibitory factor (LIF) at 1000 U/ml. Embryoid bodies from MASC or ES were formed by the hanging drop method.

Quantitative Polymerase Chain Reaction (qPCR)

Total RNA was prepared from cultured cells using the RNeasy extraction kit (Qiagen) and reverse transcribed using Superscript II reverse transcriptase (Invitrogen) according to the manufacturer's instructions. Relative quantitative PCR was performed on a 7500 Fast Real Time PCR System (Applied Biosystems) using SYBR Green PCR mix (Applied Biosystems). Mouse specific intron-spanning primer pairs used were as follows:

stra8

| | |
|---|---|
| ACAAGAGTGAGGCCCAGCAT, | (fwd, SEQ ID NO: 7) |
| CCTCTGGATTTT-CTGAGTTGCA, | (rev, SEQ ID NO: 8) |
| plzf | |
| TTTGCGACTGAGAATGCATTTAC, | (fwd, SEQ ID NO: 9) |
| ACCGCATTGATCACACACAAAG, | (rev, SEQ ID NO: 10) |
| ret | |
| GGCTGTCCCGAGATGTTTATG | (fwd, SEQ ID NO: 11) |
| GACTCAATTGCCATCCACTTGA, | (rev, SEQ ID NO: 12) |
| dazl | |
| AAATCATGCCA-AACACCGTTTT | (fwd, SEQ ID NO: 13) |
| GGCAAAGAAACTCCTGATTTCG, | (rev, SEQ ID NO: 14) |
| oct4 | |
| TTGGGCTAGAGAAGGATGTGGTT, | (fwd, SEQ ID NO: 15) |
| GGAAAAGGGACTGAGTAG-AGTGTGG, | (rev, SEQ ID NO: 16) |
| sox2 | |
| TTTTCGGTGATGCCGACTAGA, | (fwd, SEQ ID NO: 17) |
| GCGCCTAACGTACCACTAGAACTT, | (rev, SEQ ID NO: 18) |
| nanog | |
| AAGAACTCT-CCTCCATTCTGAACCT, | (fwd, SEQ ID NO: 19) |
| TGCACTTCATCCTT-TGGTTTTG, | (rev, SEQ ID NO: 20) |
| Prm1 | |
| CCGCCGCTCATACACCATA, | (fwd, SEQ ID NO: 21) |
| ACGCAGGAGTTTTGATGGACTT, | (rev, SEQ ID NO: 22) |
| Pgk2 | |
| GGACAAAGTGGATCTTAAGGGAAA, | (fwd, SEQ ID NO: 23) |
| TTGGTTATTCTTCATGGGAACGT, | (rev, SEQ ID NO: 24) |
| adam2 | |
| CTGAGTGGGCTGAGTGAACTTG | (fwd, SEQ ID NO: 25) |
| TAATTTCTCACGAG-TGCCTTCTGT, | (rev, SEQ ID NO: 26) |
| tnp1 | |
| CGGAAGAGCGTCCTGAAAAG, | (fwd, SEQ ID NO: 27) |
| CATTGCCGCATCACAAGTG, | (rev, SEQ ID NO: 28) |
| sox17 | |
| GGCCGATGAACGCCTTT, | (fwd, SEQ ID NO: 29) |
| ACGAAGGGCCGCTTCTCT, | (rev, SEQ ID NO: 30) |
| brachyury | |
| GCTGTGGCTGCGCTTCA, | (fwd, SEQ ID NO: 31) |
| GAACATCCTCCTGCCGTTCTT, | (rev, SEQ ID NO: 32) |
| dkk1 | |
| TCAAAAATATATCACACCAAAGGACA | (fwd, SEQ ID NO: 33) |
| AG, | |
| GCCCTGCGGCACAGTCT, | (rev, SEQ ID NO: 34) |
| noggin | |
| AGCTGAGGAGGAAGTTACAGATGTG, | (fwd, SEQ ID NO: 35) |
| CTAGGTCATTCC-ACGCGTACAG, | (rev, SEQ ID NO: 36) |
| zfp42(rex1) | |
| CAGCAGCTCCTGCACACAGA, | (fwd, SEQ ID NO: 37) |
| GGGCACTGATCCGCAAAC, | (rev, SEQ ID NO: 38) |
| pou3f1 | |
| GGAGCAGTTCGCCAAGCA, | (fwd, SEQ ID NO: 39) |
| TGCGAGAACACGTTACCGTAGA, | (rev, SEQ ID NO: 40) |
| gfra1 | |
| TACCACCAGCATGTCCAATGAA, | (fwd, SEQ ID NO: 41) |
| GTAGCTGTGCTTGGCTGGAACT, | (rev, SEQ ID NO: 42) |
| cd9 | |
| TGCATGCTGGGATTGTTCTTC, | (fwd, SEQ ID NO: 43) |
| GGCGGCGGCTATCTCAA, | (rev, SEQ ID NO: 44) |
| bcl6b | |
| CGCCAGGAAGTGAGTTTTTCA, | (fwd, SEQ ID NO: 45) |
| GCTCCAGCCCCGATGAG, | (rev, SEQ ID NO: 46) |
| tacstd | |
| TGCTCCAAACTGGCGTCTAA(fwd), | (Ep-CAM, SEQ ID NO: 47) |
| TCCCAGACTTGCTGTGAGTCA, | (rev, SEQ ID NO: 48) |
| esg1 | |
| GTGGGTGAAAGTTCCTGAAGACCTGA, | (fwd, SEQ ID NO: 49) |
| TGTTAGACATTCGAGAT-CCCTGTGG, | (rev, SEQ ID NO: 50) |
| gdf3 | |
| CTTCTCCCAGACCAGGGTTTTT, | (fwd, SEQ ID NO: 51) |
| CTGGAGACAGGAGCCATCTTG, | (rev, SEQ ID NO: 52) |
| itga6 | |
| ATGCAGATGGGTGGCAAGAC, | (fwd, SEQ ID NO: 53) |
| CTGCACCCCCGACTTCAC, | (rev, SEQ ID NO: 54) |
| and | |
| ddx4 | |
| (MVH)AGGACGAGATTTGATGGCTTGT, | (fwd, SEQ ID NO: 55) |
| GGCAAGAGAAAAGCT-GCAGTCT. | (rev, SEQ ID NO: 56) |

Cycle conditions were as follows: one cycle at 50° C. for 2 min followed by 1 cycle at 95° C. for 10 minutes followed by 40 cycles at 95° C. for 15 s and 60° C. for 1 minute. Specificity of PCR products was tested by dissociation curves. Threshold cycles of primer probes were normalized to the housekeeping gene GAPDH or β-actin and translated to relative values.

### Flow Cytometry

Flow cytometry was performed on either testis that had been freshly dissociated as described above or on cultured GSP or MASC following trypsinization. For fresh testicular cells, the washing step and low speed (30 g) centrifugation step was employed to remove as many of the spermatozoa (which remained in the supernatant) as possible but also likely depleted the preparation of small fragments of predominantly interstitial cells, whereas the larger fragments (in the pellet) were enzymatically dissociated for subsequent analysis. Dissociated cells were labeled with fluorescein di-D-galactopyranoside (FDG; Invitrogen) per the manufacturer's protocol. Finally, cells were filtered through a 40 μm mesh before analysis. For cell cycle analysis, cells were harvested, fixed in ethanol, incubated with 0.5 μg/ml RNAse A (Sigma-Aldrich) and 50 μg/ml propidium iodide (Sigma-Aldrich) for 3 hr at 4° C. Cytometry for c-kit was performed using PE-conjugated rat monoclonal antibody 2B8 anti-c-kit (BD Pharmingen). Cytometry was performed on a Beckman-Coulter FC500 Cytometer. Data were processed using FlowJo 7.1.2 (Tree Star, Inc.).

### Mouse VE-Cadherin Promoter

The mouse VE-Cadherin promoter sequence (generously provided by Laura Benjamin)²⁵ was subcloned into a lentiviral vector upstream of GFP (mVE-CadPr-GFP). Viral particles were produced as previously described³⁰ and used to generate MASCs with stable integration of the mVE-CadPr-GFP reporter construct. Mouse VE-CadPr-GFP MASCs were injected into NOD-SCID mice to form teratomas after 3-4 weeks and contribution of GFP⁺(VE-Cadherin⁺) cells to the vasculature was assessed by confocal microscopy as described above.

### Mouse Embryo Chimeras

Either unlabeled GPR125^{lacZ/lacZ} or Rosa-βgal MASCs or GPR125^{lacZ/lacZ} MASCs that had been previously stably transduced with either GFP or mCherry under control or the PGK promoter by lentivirus³⁰⁻³¹ and then cloned were employed for chimerism experiments, using previously described protocols¹⁴. In brief, cells were injected into E3.5 C57B16 blastocysts and implanted into surrogate pseudopregnant female mice. Surrogate mothers were sacrificed and embryos harvested at embryonic days 10.5 to 18.5 for analysis by confocal microscopy (for GFP or mCherry labeled clones) or whole mount X-gal staining.

### Spermatogonial Stem Cell Transplantation and Analysis of Recipient Testes

Adult C57BL/6 male mice were administered with a single i.p. injection of busulfan (40 mg/kg body weight) at 5-6 weeks of age and used as recipients 4-8 weeks later. GPR125^{lacZ/lacZ} GSP cultures stably transduced with GFP driven by the PGK promoter (delivered by lentivirus) were transplanted. Cultured cells were dissociated using 0.05% trypsin/EDTA and resuspended in GSP culture medium containing DNase I (30 μg/ml) at a concentration of 8×10⁶ cells/ml. Viability, evaluated by trypan blue exclusion, was higher than 90%. Approximately 8 μl (corresponding to 3-5×10⁴ cells) of donor cell suspension were transplanted in each testis via efferent ducts³². Addition of trypan blue to cell suspensions revealed 70-95% filling of seminiferous tubules. Two to three months after transplantation, recipient testes were collected, detunicated and analyzed fresh for GFP expression fluorescent stereo or confocal microscopy. Each testis was then divided in fragments and processed for additional fluorescent imaging or for X-gal staining. Samples for whole-mount X-gal staining were fixed in 4% paraformaldehyde/PBS for 2 h at 4° C. After washes in PBS, they were incubated overnight at 4° C. in LacZ buffer (0.2 M sodium phosphate [pH 7.3], 2 mM MgCl₂, 0.02% (v/v) NP-40, 0.01% (v/v) sodium deoxycholate, 20 mM potassium ferricyanide, and 20 mM potassium ferrocyanide). The next day, staining was performed by incubating testes in LacZ staining solution (LacZ buffer containing 1 mg/ml of X-gal) for 4 h at 37° C. After X-gal staining, samples were analyzed for LacZ expression by light microscopy. Preservation of GFP fluorescence even after X-gal staining allowed concomitant visualization of GFP and X-gal in the same cells. This tissue was then processed for paraffin embedding, sectioned and stained with Nuclear Fast Red. For optimal GFP preservation, other tubule fragments were fixed overnight in 4% paraformaldehyde/PBS at 4° C., washed and then image as whole tubules on the confocal microscope or cryopreserved in OCT and sectioned.

### Statistical Analysis

Image analysis of X-gal stained fields of GPR125^{+/lacZ} testis was performed as follows. Color images were captured using an Olympus microscope converted to grayscale in Adobe Photoshop 7.0 (San Jose, Calif.). ImageJ 1.36b (NIH) was used to perform thresholding (uniformly for all fields analyzed) and measurement of stained area within transverse cross-sections of tubules categorized as stage 1V-V or stage VII-VIII. The Wilcoxon test for non-parametric data was performed using SPSS 9.0 (Chicago, Ill.).

### REFERENCES FOR EXAMPLE 1

1. Guan, K. et al. Pluripotency of spermatogonial stem cells from adult mouse testis. Nature 440, 1199-1203 (2006).
2. Kanatsu-Shinohara, M. & Shinohara, T. The germ of pluripotency. Nat. Biotechnol. 24, 663-664 (2006).
3. Valenzuela, D. M. et al. High-throughput engineering of the mouse genome coupled with high-resolution expression analysis. Nat. Biotechnol. 21, 652-659 (2003).
4. Oakberg, E. F. A description of spermiogenesis in the mouse and its use in analysis of the cycle of the seminiferous epithelium and germ cell renewal. Am J Anat. 99, 391-413 (1956).
5. Fiering, S. N. et al. Improved FACS-Gal: flow cytometric analysis and sorting of viable eukaryotic cells expressing reporter gene constructs. Cytometry 12, 291-301 (1991).
6. Kuroda, N. et al. Distribution and role of CD34-positive stromal cells and myofibroblasts in human normal testicular stroma. Histol. Histopathol. 19, 743-751 (2004).
7. Schaefer, B. C., Schaefer, M. L., Kappler, J. W., Marrack, P. & Kedl, R. M. Observation of antigen-dependent CD8+T-cell/dendritic cell interactions in vivo. Cell Immunol. 214, 110-122 (2001).
8. Enders, G. C. & May, J. J. Developmentally regulated expression of a mouse gems cell nuclear antigen examined from embryonic day 11 to adult in male and female mice. Dev. Biol. 163, 331-340 (1994).
9. Schrans-Stassen, B. H., Saunders, P. T., Cooke, H. J. & de Rooij, D. G. Nature of the spermatogenic arrest in Dazl −/− mice. Biol. Reprod. 65, 771-776 (2001).
10. Tanaka, S. S. et al. The mouse homolog of Drosophila Vasa is required for the development of male germ cells. Genes Dev. 14, 841-853 (2000).
11. Costoya, J. A. et al. Essential role of Plzf in maintenance of spermatogonial stem cells. Nat. Genet. 36, 653-659 (2004).
12. Buaas, F. W. et al. Plzf is required in adult male germ cells for stem cell self-renewal. Nat. Genet. 36, 647-652 (2004).
13. Brinster, R. L. & Zimmermann, J. W. Spermatogenesis following male germ-cell transplantation. Proc Natl. Acad. Sci. U S. A 91, 11298-11302 (1994).
14. Kanatsu-Shinohara, M. et al. Generation of pluripotent stem cells from neonatal mouse testis. Cell 119, 1001-1012 (2004).
15. Schatten, G., Smith, J., Navara, C., Park, J. H. & Pedersen, R. Culture of human embryonic stem cells. Nat. Methods 2, 455-463 (2005).
16. Friedrich, G. & Soriano, P. Promoter traps in embryonic stem cells: a genetic screen to identify and mutate developmental genes in mice. Genes Dev. 5, 1513-1523 (1991).
17. Reijo, R. A. et al. DAZ family proteins exist throughout male germ cell development and transit from nucleus to cytoplasm at meiosis in humans and mice. Biol. Reprod. 63, 1490-1496 (2000).
18. Ehmcke, J., Hubner, K., Scholer, H. R. & Schlatt, S. Spermatogonia: origin, physiology and prospects for conservation and manipulation of the male germ line. Reprod. Fertil. Dev. 18, 7-12 (2006).
19. Wang, P. J., Page, D. C. & McCarrey, J. R. Differential expression of sex-linked and autosomal germ-cell-specific genes during spermatogenesis in the mouse. Hum. Mol. Genet. 14, 2911-2918 (2005).
20. Ryu, B. Y., Orwig, K. E., Kubota, H., Avarbock, M. R. & Brinster, R. L. Phenotypic and functional characteristics of spermatogonial stem cells in rats. Dev. Biol. 274, 158-170 (2004).
21. Kanatsu-Shinohara, M., Toyokuni, S. & Shinohara, T. CD9 is a surface marker on mouse and rat male germline stem cells. Biol. Reprod. 70, 70-75 (2004).
22. Shinohara, T., Avarbock, M. R. & Brinster, R. L. beta1- and alpha6-integrin are surface markers on mouse spermatogonial stem cells. Proc Natl. Acad. Sci. U.S. A 96, 5504-5509 (1999).
23. Seydoux, G. & Braun, R. E. Pathway to totipotency: lessons from germ cells. Cell 127, 891-904 (2006).
24. Keller, G. Embryonic stem cell differentiation: emergence of a new era in biology and medicine. Genes Dev. 19, 1129-1155 (2005).
25. Sun, J. F. et al. Microvascular patterning is controlled by fine-tuning the Akt signal. Proc Natl. Acad. Sci. U. S. A 102, 128-133 (2005).
26. Simon, A. & Frisen, J. From stem cell to progenitor and back again. Cell 128, 825-826 (2007).
27. Jiang, Y. et al. Pluripotency of mesenchymal stem cells derived from adult marrow. Nature 418, 41-49 (2002).
28. Baba, S. et al. Generation of Cardiac and Endothelial Cells from Neonatal Mouse Testis-derived Multipotent Germline Stem Cells. Stem Cells (2007).
29. Kanatsu-Shinohara, M. et al. Long-term proliferation in culture and germline transmission of mouse male germline stem cells. Biol. Reprod. 69, 612-616 (2003).
30. Naldini, L. et al. In vivo gene delivery and stable transduction of nondividing cells by a lentiviral vector. Science 272, 263-267 (1996).
31. Shaner, N. C. et al. Improved monomeric red, orange and yellow fluorescent proteins derived from Discosoma sp. red fluorescent protein. Nat. Biotechnol. 22, 1567-1572 (2004).
32. Ogawa, T., Arechaga, J. M., Avarbock, M. R. & Brinster, R. L. Transplantation of testis germinal cells into mouse seminiferous tubules. Int. J Dev. Biol. 41, 111-122 (1997).

### Example 2

### GPR125 As a Cancer Stem Cell Marker

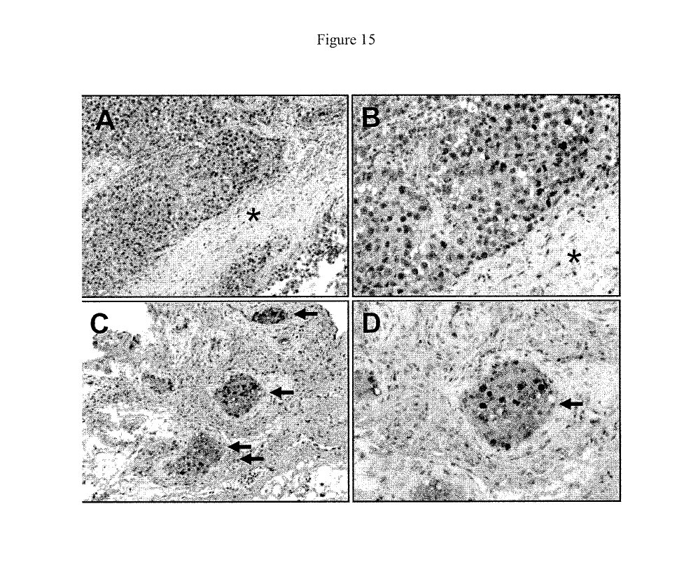

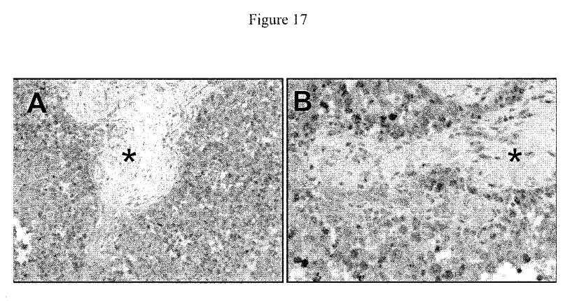

Immunostaining for GPR125 was performed on human testicular germ cell tumors obtained from three separate patients. Paraffin embedded tissue was stained using a polyclonal peptide antibody against GPR125. FIG. 15 shows the results from patient 1, FIG. 16 shows the results from patient 2, and FIG. 17 shows the results from patient 3. Positive (dark) staining was seen in abnormal seminiferous tubules (indicated by arrows in the figures) adjacent to the tumor and in clusters of tumor cells, but was not seen in intervening fibrous stroma (indicated by asterisks in the figures). This data suggests that GPR125 may be a cancer stem cell marker.

### Example 3

### GPR125 As a Marker of Skin, Intestinal and Neural Stem Cells

GPR125 expression was analyzed in various other tissues. The pattern of expression seen suggested that GPR125 may be a stem cell marker in the skin (including hair follicles), the intestine, and the nervous system (including the retina). Xgal staining of frozen sections was performed to detect GPR125-lacZ expression in the transgenic mice described in Example 1 at various different ages. Expression (as seen by Xgal staining) was seen in the skin of GPR125-lacZ mice at various embryonic stages and in newborn mice. The pattern of expression seen in the newborn mice suggested co-localization with the putative bulge stem cells (stem cells in the bulge region of hair follicles—data not shown.

The staining pattern for GPR125-lacZ was analyzed in relation to the pattern of alpha6-integrin staining. First, Xgal staining of frozen sections was performed to detect GPR125-lacZ expression in adult skin and then the same sections were immunostained for alpha6-integrin, which is expressed at high levels on bulge stem cells (stem cells in the bulge region of hair follicles). The staining revealed that GPR125-lacZ and alpha6-integrin were co-localized, suggesting that GPR125 may be a stem cell marker in the skin, and in particular a marker of bulge stem cells—the stem cells in the bulge region of hair follicles.

GPR125 expression was also analyzed in the mouse eye and in cultured retinospheres. Xgal staining of frozen sections was performed to detect GPR125-lacZ expression in the lacZ transgenic mice described in Example 1 at various ages. X-gal staining in the ciliary marginal zone was seen, suggesting co-localization of GPR125 with putative retinal stem cells. Ciliary marginal zone cells were microdissected and cultured for approximately 1 week in retinal stem cell medium. Retinospheres were cultured from GPR125LacZ knock-in mice, rosa26-LacZ mice, and from wild type C57b1/6j mice. Xgal staining was performed to detect GPR125-lacZ expression. Long-term expression of GPR125-lacZ within the retinospheres was seen, consistent with the positive cells having a retinal stem cell phenotype.

GPR125 expression was also analyzed in the mouse brain. Xgal staining of frozen sections was performed to detect GPR125-lacZ expression at various ages. Xgal staining was seen in the subventricular zone, suggesting co-localization with putative neural stem cells. Subventricular zone cells were microdissected from both GPR125LacZ knock-in mice and wild type C57b1/6j mice and cultured for approximately 1 month in neural stem cell medium. Neurospheres were formed. Xgal staining was performed to detect GPR125-lacZ expression. Maintenance of long-term GPR125-lacZ expression within the neurospheres was seen, consistent with the cells having a putative neural stem cell phenotype.

GPR125 expression was also analyzed in the adult mouse small intestine. Xgal staining of frozen sections was performed to detect GPR125-lacZ expression. Expression was seen in the base of the crypts, suggesting co-localization with the putative intestinal stem cells.

### Example 4

### GPR125-Positive Stem or Progenitor Cells in Humans

To determine whether GPR125 is expressed in a similar location in the human testis as in mouse, paraffin section from human testes were stained with rabbit polyclonal anti-GPR125 antibody. Human samples consisted of testicular tissue taken from patients with infertility or who had undergone orchiectomy for testicular germ cell tumor. GPR125 staining was seen only within the seminiferous tubules or within the tumor tissue (data not shown), consistent with the findings in the mouse that GPR125 in the testis is restricted to the germ cells within the seminiferous tubules. This data suggests that GPR125 is a stem and/or progenitor cell marker in human tissues.

Cells were cultured from normal human testis using methods as described herein. Human SPCs were isolated from short term cultures of fresh normal human testicular tissue by incubating the cells with an antibody to the stem cell marker alpha6 integrin (rat anti-alpha6 integrin), followed by selection using magnetic beads conjugated to anti-rat antibody. After 10 days of further culture on feeder cells, stem cell-like colonies were seen in the alpha6 integrin-positive fraction but not the alpha6-negative fraction of cells. Positive staining with anti-VASA antibody confirmed the majority of cells to be germ cells in the human SPC cultures, while positive staining with anti-plzf (a stem cell marker) confirmed the majority of those germ cells to be undifferentiated spermatogonia.

1. A method for enriching stem or progenitor cells from a mixed population of cells, comprising:
(a) contacting a mixed population of cells with an agent that binds to GPR125, and
(b) separating the cells bound by the agent from cells that are not bound by the agent,
wherein the cells bound by the agent comprise a subpopulation of the mixed population of cells that is enriched for stem or progenitor cells.

2-3. (canceled)

4. The method of claim 1, wherein the mixed population of cells are mammalian cells.

5-6. (canceled)

7. The method of claim 4, wherein the mammalian cells are human cells.

8. The method of claim 1, wherein the mixed population of cells comprise testis, skin, intestine or neural cells.

9. (canceled)

10. The method of claim 1, wherein the stem or progenitor cells are selected from the group consisting of multipotent adult spermatogonial-derived stem cells (MASCs), spermatogonial stem or progenitor cell, skin stem or progenitor cells, intestinal stem or progenitor cells and neural stem or progenitor cells.

11. (canceled)

12. The method of claim 1, wherein the agent is an antibody.

13-17. (canceled)

18. The method of claim 1, wherein the agent is an antibody and the step of separating is performed using immuno-affinity purification.

19. (canceled)

20. The method of claim 1, wherein the agent is an antibody labeled with a fluorescent moiety and the step of separating the subpopulation of cells that are bound by the agent from the subpopulation of cells that are not bound by the agent is performed using fluorescence activated cell sorting (FACS).

21. A method for detecting stem or progenitor cells in a cell or tissue sample, comprising:
(a) contacting a cell or tissue sample with an agent that binds to GPR125 protein or an agent that binds to GPR125 mRNA, and
(b) determining whether the agent has bound to the cell or tissue sample,
wherein binding indicates the presence of stem or progenitor cells in the cell or tissue sample.

22-25. (canceled)

26. The method of claim 21, wherein the cell or tissue sample is derived from, testis, skin, intestine or neural tissue.

27. (canceled)

28. The method of claim 21, wherein the stem or progenitor cells are selected from the group consisting of multipotent adult spermatogonial-derived stem cells (MASCs), spermatogonial stem or progenitor cells, skin stem or progenitor cells, intestinal stem or progenitor cells and neural stem or progenitor cells.

29. (canceled)

30. The method of claim 21, wherein the agent is an antibody.

31-36. (canceled)

37. An isolated preparation consisting essentially of GPR125-positive stem or progenitor cells.

38-40. (canceled)

41. The isolated preparation of claim 37, wherein the GPR125-positive stem or progenitor cells are spermatogonial stem or progenitor cells, and wherein the GPR125-positive stem or progenitor cells express at least one gene selected from the group consisting of DAZL, VASA, integrin alpha 6, Ep-CAM, CD9, GFRa1, glial derived neurotrophic factor (GDNF) and Stra8.

42. The isolated preparation of claim 37, wherein the stem or progenitor cells comprise MASCs, and wherein the MASCs express GPR125 and at least one gene selected from the group consisting of oct4, nanog, and sox2.

43-81. (canceled)

82. A method of reconstituting or supplementing spermatogenesis in a subject in need thereof, comprising administering to the subject GPR125-positive spermatogonial stem or progenitor cells.

83. The method of claim 82, wherein the subject is infertile or has reduced fertility.

84-88. (canceled)

89. The method of claim 82, wherein the GPR125-positive spermatogonial stem or progenitor cells are administered by direct injection into the testis.

90. The method of claim 82, wherein the subject is a mammal selected from the group consisting of primates, rodents, ovine species, bovine species, porcine species, equine species, feline species and canine species.

91. (canceled)

92. The method of claim 82, wherein the subject is a human.

93-121. (canceled)

122. The method of claim 1, wherein the stem or progenitor cells are cancer stem cells.

123. The method of claim 21, wherein the stem or progenitor cells are cancer stem cells.

124. The isolated preparation of claim 37, wherein the stem or progenitor cells are cancer stem cells.


SEQUENCE LISTING (56 sequences)

SEQ ID NO: 1 - protein, 1321 aa, Homo sapiens
MEPPGRRRGRAQPPLLLPLSLLALLALLGGGGGGGAAALPAGCKHDGRPRGAGRAAGAAEGKVVCSSLELAQVLPPDTLPNRTVTLILSNNKISELKNGSFSGLSLLERLDLRNNLISSIDPGAFWGLSSLKRLDLTNNRIGCLNADIFRGLTNLVRLNLSGNLFSSLSQGTFDYLASLRSLEFQTEYLLCDCNILWMHRWVKEKNITVRDTRCVYPKSLQAQPVTGVKQELLTCDPPLELPSFYMTPSHRQVVFEGDSLPFQCMASYIDQDMQVLWYQDGRIVETDESQGIFVEKNMIHNCSLIASALTISNIQAGSTGNWGCHVQTKRGNNTRTVDIVVLESSAQYCPPERVVNNKGDFRWPRTLAGITAYLQCTRNTHGSGIYPGNPQDERKAWRRCDRGGFWADDDYSRCQYANDVTRVLYMFNQMPLNLTNAVATARQLLAYTVEAANFSDKMDVIFVAEMIEKFGRFTKEEKSKELGDVMVDIASNIMLADERVLWLAQREAKACSRIVQCLQRIATYRLAGGAHVYSTYSPNIALEAYVIKSTGFTGMTCTVFQKVAASDRTGLSDYGRRDPEGNLDKQLSFKCNVSNTFSSLALKNTIVEASIQLPPSLFSPKQKRELRPTDDSLYKLQLIAFRNGKLFPATGNSTNLADDGKRRTVVTPVILTKIDGVNVDTHHIPVNVTLRRIAHGADAVAARWDFDLLNGQGGWKSDGCHILYSDENITTIQCYSLSNYAVLMDLTGSELYTQAASLLHPVVYTTAIILLLCLLAVIVSYIYHHSLIRISLKSWHMLVNLCFHIFLTCVVFVGGITQTRNASICQAVGIILHYSTLATVLWVGVTARNIYKQVTKKAKRCQDPDEPPPPPRPMLRFYLIGGGIPIIVCGITAAANIKNYGSRPNAPYCWMAWEPSLGAFYGPASFITFVNCMYFLSIFIQLKRHPERKYELKEPTEEQQRLAANENGEINHQDSMSLSLISTSALENEHTFHSQLLGASLTLLLYVALWMFGALAVSLYYPLDLVFSFVFGATSLSFSAFFVVHHCVNREDVRLAWIMTCCPGRSSYSVQVNVQPPNSNGTNGEAPKCPNSSAESSCTNKSASSFKNSSQGCKLTNLQAAAAQCHANSLPLNSTPQLDNSLTEHSMDNDIKMHVAPLEVQFRTNVHSSRHHKNRSKGHRASRLTVLREYAYDVPTSVEGSVQNGLPKSRLGNNEGHSRSRRAYLAYRERQYNPPQQDSSDACSTLPKSSRNFEKPVSTTSKKDALRKPAVVELENQQKSYGLNLAIQNGPIKSNGQEGPLLGTDSTGNVRTGLWKHETTV

SEQ ID NO: 2 - DNA, 4576 bp, Homo sapiens
cggcagaggaggaggaggaggcggtgctcgcgccgggcggtagagcggcgctgggacccatgcggccgtgacccccggctccctagaggcccagcgcagccgcagcggacaaaggagcatgtccgcgccggggaaggcccgtcctccggccgccataaggctccggtcgccgctgggcccgcgccgcgctcctgcccgcccgggctccggggcggcccgctaggccagtgcgccgccgctcgccccgcaggccccggcccgcagcatggagccacccggacgccggcggggccgcgcgcagccgccgctgttgctgccgctctcgctgttagcgctgctcgcgctgctgggaggcggcggcggcggcggcgccgcggcgctgcccgccggctgcaagcacgatgggcggccccgaggggctggcagggcggcgggcgccgccgagggcaaggtggtgtgcagcagcctggaactcgcgcaggtcctgcccccagatactctgcccaaccgcacggtcaccctgattctgagtaacaataagatatccgagctgaagaatggctcattttctgggttaagtctccttgaaagattggacctccgaaacaatcttattagtagtatagatccaggtgccttctggggactgtcatctctaaaaagattggatctgacaaacaatcgaataggatgtctgaatgcagacatatttcgaggactcaccaatctggttcggctaaacctttcggggaatttgttttcttcattatctcaaggaacttttgattatcttgcgtcattacggtctttggaattccagactgagtatcttttgtgtgactgtaacatactgtggatgcatcgctgggtaaaggagaagaacatcacggtacgggataccaggtgtgtttatcctaagtcactgcaggcccaaccagtcacaggcgtgaagcaggagctgttgacatgcgaccctccgcttgaattgccgtctttctacatgactccatctcatcgccaagttgtgtttgaaggagacagccttcctttccagtgcatggcttcatatattgatcaggacatgcaagtgttgtggtatcaggatgggagaatagttgaaaccgatgaatcgcaaggtatttttgttgaaaagaacatgattcacaactgctccttgattgcaagtgccctaaccatttctaatattcaggctggatctactggaaattggggctgtcatgtccagaccaaacgtgggaataatacgaggactgtggatattgtggtattagagagttctgcacagtactgtcctccagagagggtggtaaacaacaaaggtgacttcagatggcccagaacattggcaggcattactgcatatctgcagtgtacgcggaacacccatggcagtgggatatatcccggaaacccacaggatgagagaaaagcttggcgcagatgtgatagaggtggcttttgggcagatgatgattattctcgctgtcagtatgcaaatgatgtcactagagttctttatatgtttaatcagatgcccctcaatcttaccaatgccgtggcaacagctcgacagttactggcttacactgtggaagcagccaacttttctgacaaaatggatgttatatttgtggcagaaatgattgaaaaatttggaagatttaccaaggaggaaaaatcaaaagagctaggtgacgtgatggttgacattgcaagtaacatcatgttggctgatgaacgtgtcctgtggctggcgcagagggaagctaaagcctgcagtaggattgtgcagtgtcttcagcgcattgctacctaccggctagccggtggagctcacgtttattcaacatattcacccaatattgctctggaagcttatgtcatcaagtctactggcttcacggggatgacctgtaccgtgttccagaaagtggcagcctctgatcgtacaggactttcggattatgggaggcgggatccagagggaaacctggataagcagctgagctttaagtgcaatgtttcaaatacattttcgagtctggcactaaagaatactattgtggaggcttctattcagcttcctccttcccttttctcaccaaagcaaaaaagagaactcagaccaactgatgactctctttacaagcttcaactcattgcattccgcaatggaaagctttttccagccactggaaattcaacaaatttggctgatgatggaaaacgacgtactgtggttacccctgtgattctcaccaaaatagatggtgtgaatgtagatacccaccacatccctgttaatgtgacactgcgtcgaattgcacatggagcagatgctgttgcagcccggtgggatttcgatttgctgaacggacaaggaggctggaagtcagatgggtgccatatactctattcagatgaaaatatcactacgattcagtgctactcccttagtaactatgcagttttaatggatttgacgggatctgaactatacacccaggcggccagcctcctgcatcctgtggtttatactaccgctatcattctcctcttatgtctcttagccgtcattgtcagttacatataccatcacagtttgattagaatcagcctcaagagctggcacatgcttgtgaacttgtgctttcatattttcctaacctgtgtggtctttgtgggaggaataacccagactaggaatgccagcatctgccaagcagttgggataattcttcactattccacccttgccacagtactatgggtaggagtgacagctcgaaatatctacaaacaagtcactaaaaaagctaaaagatgccaggatcctgatgaaccaccacctccaccaagaccaatgctcagattttacctgattggtggtggtatccccatcattgtttgcggcataactgcagcagcgaacattaagaattacggcagtcggccaaacgcaccctattgctggatggcatgggaaccctccttgggagccttctatgggccagccagcttcatcacttttgtaaactgcatgtactttctgagcatatttattcagttgaaaagacaccctgagcgcaaatatgagcttaaggagcccacggaggagcaacagagattggcagccaatgaaaatggcgaaataaatcatcaggattcaatgtctttgtctctgatttctacatcagccttggaaaatgagcacacttttcattctcagctcttgggggccagccttactttgctcttatatgttgcactgtggatgtttggggctttggctgtttctttgtattaccctttggacttggtttttagcttcgtttttggagccacaagtttaagcttcagtgcgttcttcgtggtccaccattgtgttaatagggaggatgttagacttgcgtggatcatgacttgctgcccaggacggagctcgtattcagtgcaagtcaacgtccagccccccaactctaatgggacgaatggagaggcacccaaatgccccaatagcagtgcggagtcttcatgcacaaacaaaagtgcttcaagcttcaaaaattcctcccagggctgcaaattaacaaacttgcaggcggctgcagctcagtgccatgccaattctttacctttgaactccacccctcagcttgataatagtctgacagaacattcaatggacaatgatattaaaatgcacgtggcgcctttagaagttcagtttcgaacaaatgtgcactcaagccgccaccataaaaacagaagtaaaggacaccgggcaagccgactcacagtcctgagagaatatgcctacgatgtcccaacgagcgtggaaggaagcgtgcagaacggcttacctaaaagccggctgggcaataacgaaggacactcgaggagccgaagagcttatttagcctacagagagagacagtacaacccaccccagcaagacagcagcgatgcttgtagcacacttcccaaaagtagcagaaattttgaaaagccagtttcaaccactagtaaaaaagatgcgttaaggaagccagctgtggttgaacttgaaaatcagcaaaaatcttatggcctcaacttggccattcagaatggaccaattaaaagcaatgggcaggagggacccttgctcggtaccgatagcactggcaatgttaggactggattatggaaacacgaaactactgtgtaacattgctgggcttcctaggcagaaattcatataaactgtgatactcacattccttgaagctatgagcatttaaaaactgtttacagccaccatagggattcaaaagaatttggaataaactttgaagttttggattttacttatttttatccccaaattgttgctattttttaggatctgaaacaaaatctttctaaaacattgttttagttgtcaaagcaccaacaggacattttgggatgtgaaatgtaatttcttggaatctgtaatttgtacttaatatttcaggcttgtatttaatataataaataggtgtttgttattgtgtcaaaaaaaaaaaaaaaa

SEQ ID NO: 3 - DNA, 22 bp, Artificial Sequence
  Description of Artificial Sequence Synthetic primer
atgttagcttaaatggactgtc

SEQ ID NO: 4 - DNA, 22 bp, Artificial Sequence
  Description of Artificial Sequence Synthetic primer
gtctgtcctagcttcctcactg

SEQ ID NO: 5 - DNA, 22 bp, Artificial Sequence
  Description of Artificial Sequence Synthetic primer
tcattctcagtattgttttgcc

SEQ ID NO: 6 - DNA, 21 bp, Artificial Sequence
  Description of Artificial Sequence Synthetic primer
atagtaaatcccaaagctcac

SEQ ID NO: 7 - DNA, 20 bp, Artificial Sequence
  Description of Artificial Sequence Synthetic primer
acaagagtgaggcccagcat

SEQ ID NO: 8 - DNA, 22 bp, Artificial Sequence
  Description of Artificial Sequence Synthetic primer
cctctggattttctgagttgca

SEQ ID NO: 9 - DNA, 23 bp, Artificial Sequence
  Description of Artificial Sequence Synthetic primer
tttgcgactgagaatgcatttac

SEQ ID NO: 10 - DNA, 22 bp, Artificial Sequence
  Description of Artificial Sequence Synthetic primer
accgcattgatcacacacaaag

SEQ ID NO: 11 - DNA, 21 bp, Artificial Sequence
  Description of Artificial Sequence Synthetic primer
ggctgtcccgagatgtttatg

SEQ ID NO: 12 - DNA, 22 bp, Artificial Sequence
  Description of Artificial Sequence Synthetic primer
gactcaattgccatccacttga

SEQ ID NO: 13 - DNA, 22 bp, Artificial Sequence
  Description of Artificial Sequence Synthetic primer
aaatcatgccaaacaccgtttt

SEQ ID NO: 14 - DNA, 22 bp, Artificial Sequence
  Description of Artificial Sequence Synthetic primer
ggcaaagaaactcctgatttcg

SEQ ID NO: 15 - DNA, 23 bp, Artificial Sequence
  Description of Artificial Sequence Synthetic primer
ttgggctagagaaggatgtggtt

SEQ ID NO: 16 - DNA, 25 bp, Artificial Sequence
  Description of Artificial Sequence Synthetic primer
ggaaaagggactgagtagagtgtgg

SEQ ID NO: 17 - DNA, 21 bp, Artificial Sequence
  Description of Artificial Sequence Synthetic primer
ttttcggtgatgccgactaga

SEQ ID NO: 18 - DNA, 24 bp, Artificial Sequence
  Description of Artificial Sequence Synthetic primer
gcgcctaacgtaccactagaactt

SEQ ID NO: 19 - DNA, 25 bp, Artificial Sequence
  Description of Artificial Sequence Synthetic primer
aagaactctcctccattctgaacct

SEQ ID NO: 20 - DNA, 22 bp, Artificial Sequence
  Description of Artificial Sequence Synthetic primer
tgcacttcatcctttggttttg

SEQ ID NO: 21 - DNA, 19 bp, Artificial Sequence
  Description of Artificial Sequence Synthetic primer
ccgccgctcatacaccata

SEQ ID NO: 22 - DNA, 22 bp, Artificial Sequence
  Description of Artificial Sequence Synthetic primer
acgcaggagttttgatggactt

SEQ ID NO: 23 - DNA, 24 bp, Artificial Sequence
  Description of Artificial Sequence Synthetic primer
ggacaaagtggatcttaagggaaa

SEQ ID NO: 24 - DNA, 23 bp, Artificial Sequence
  Description of Artificial Sequence Synthetic primer
ttggttattcttcatgggaacgt

SEQ ID NO: 25 - DNA, 22 bp, Artificial Sequence
  Description of Artificial Sequence Synthetic primer
ctgagtgggctgagtgaacttg

SEQ ID NO: 26 - DNA, 24 bp, Artificial Sequence
  Description of Artificial Sequence Synthetic primer
taatttctcacgagtgccttctgt

SEQ ID NO: 27 - DNA, 20 bp, Artificial Sequence
  Description of Artificial Sequence Synthetic primer
cggaagagcgtcctgaaaag

SEQ ID NO: 28 - DNA, 19 bp, Artificial Sequence
  Description of Artificial Sequence Synthetic primer
cattgccgcatcacaagtg

SEQ ID NO: 29 - DNA, 17 bp, Artificial Sequence
  Description of Artificial Sequence Synthetic primer
ggccgatgaacgccttt

SEQ ID NO: 30 - DNA, 18 bp, Artificial Sequence
  Description of Artificial Sequence Synthetic primer
acgaagggccgcttctct

SEQ ID NO: 31 - DNA, 17 bp, Artificial Sequence
  Description of Artificial Sequence Synthetic primer
gctgtggctgcgcttca

SEQ ID NO: 32 - DNA, 21 bp, Artificial Sequence
  Description of Artificial Sequence Synthetic primer
gaacatcctcctgccgttctt

SEQ ID NO: 33 - DNA, 28 bp, Artificial Sequence
  Description of Artificial Sequence Synthetic primer
tcaaaaatatatcacaccaaaggacaag

SEQ ID NO: 34 - DNA, 17 bp, Artificial Sequence
  Description of Artificial Sequence Synthetic primer
gccctgcggcacagtct

SEQ ID NO: 35 - DNA, 25 bp, Artificial Sequence
  Description of Artificial Sequence Synthetic primer
agctgaggaggaagttacagatgtg

SEQ ID NO: 36 - DNA, 22 bp, Artificial Sequence
  Description of Artificial Sequence Synthetic primer
ctaggtcattccacgcgtacag

SEQ ID NO: 37 - DNA, 20 bp, Artificial Sequence
  Description of Artificial Sequence Synthetic primer
cagcagctcctgcacacaga

SEQ ID NO: 38 - DNA, 18 bp, Artificial Sequence
  Description of Artificial Sequence Synthetic primer
gggcactgatccgcaaac

SEQ ID NO: 39 - DNA, 18 bp, Artificial Sequence
  Description of Artificial Sequence Synthetic primer
ggagcagttcgccaagca

SEQ ID NO: 40 - DNA, 22 bp, Artificial Sequence
  Description of Artificial Sequence Synthetic primer
tgcgagaacacgttaccgtaga

SEQ ID NO: 41 - DNA, 22 bp, Artificial Sequence
  Description of Artificial Sequence Synthetic primer
taccaccagcatgtccaatgaa

SEQ ID NO: 42 - DNA, 22 bp, Artificial Sequence
  Description of Artificial Sequence Synthetic primer
gtagctgtgcttggctggaact

SEQ ID NO: 43 - DNA, 21 bp, Artificial Sequence
  Description of Artificial Sequence Synthetic primer
tgcatgctgggattgttcttc

SEQ ID NO: 44 - DNA, 17 bp, Artificial Sequence
  Description of Artificial Sequence Synthetic primer
ggcggcggctatctcaa

SEQ ID NO: 45 - DNA, 21 bp, Artificial Sequence
  Description of Artificial Sequence Synthetic primer
cgccaggaagtgagtttttca

SEQ ID NO: 46 - DNA, 17 bp, Artificial Sequence
  Description of Artificial Sequence Synthetic primer
gctccagccccgatgag

SEQ ID NO: 47 - DNA, 20 bp, Artificial Sequence
  Description of Artificial Sequence Synthetic primer
tgctccaaactggcgtctaa

SEQ ID NO: 48 - DNA, 21 bp, Artificial Sequence
  Description of Artificial Sequence Synthetic primer
tcccagacttgctgtgagtca

SEQ ID NO: 49 - DNA, 26 bp, Artificial Sequence
  Description of Artificial Sequence Synthetic primer
gtgggtgaaagttcctgaagacctga

SEQ ID NO: 50 - DNA, 25 bp, Artificial Sequence
  Description of Artificial Sequence Synthetic primer
tgttagacattcgagatccctgtgg

SEQ ID NO: 51 - DNA, 22 bp, Artificial Sequence
  Description of Artificial Sequence Synthetic primer
cttctcccagaccagggttttt

SEQ ID NO: 52 - DNA, 21 bp, Artificial Sequence
  Description of Artificial Sequence Synthetic primer
ctggagacaggagccatcttg

SEQ ID NO: 53 - DNA, 20 bp, Artificial Sequence
  Description of Artificial Sequence Synthetic primer
atgcagatgggtggcaagac

SEQ ID NO: 54 - DNA, 18 bp, Artificial Sequence
  Description of Artificial Sequence Synthetic primer
ctgcacccccgacttcac

SEQ ID NO: 55 - DNA, 22 bp, Artificial Sequence
  Description of Artificial Sequence Synthetic primer
aggacgagatttgatggcttgt

SEQ ID NO: 56 - DNA, 22 bp, Artificial Sequence
  Description of Artificial Sequence Synthetic primer
ggcaagagaaaagctgcagtct